(19) United States
(12) Patent Application Publication
(10) Pub. No.: US 2026/0242503 A1
(43) Pub. Date: Aug. 20, 2026

(54) Methods of Treating High Risk Multiple Myeloma

(71) Applicant: Janssen Biotech, Inc., Horsham, PA (US)

(72) Inventors: Tahamtan Ahmadi, Rydal, PA (US); Christopher Chiu, Warren, NJ (US); Ming Qi, Phoenixville, PA (US); Amy Sasser, Doylestown, PA (US); Jordan Schecter, Livingston, NJ (US)

(21) Appl. No.: 19/666,147

(22) Filed: May 1, 2026

Related U.S. Application Data
(63) Continuation of application No. 18/175,345, filed on Feb. 27, 2023.
Application No. 18/175,345 is a division of application No. 16/177,239, filed on Oct. 31, 2018, now Pat. No. 11,618,787.
(60) Provisional application No. 62/579,234, filed on Oct. 31, 2017.

(51) Int. Cl. C07K 16/28; A61K 9/00; A61K 31/4439; A61K 31/454; A61K 31/495; A61K 31/573; A61K 31/69; A61K 39/00; A61K 39/395; A61P 35/00

(52) U.S. Cl. CPC C07K 16/2896; A61K 9/0019; A61K 31/4439; A61K 31/454; A61K 31/573; A61K 31/69; A61K 39/3955; A61P 35/00; A61K 31/495; A61K 2039/54; A61K 2039/545; C07K 2317/34; C07K 2317/56; C07K 2317/565

(57) ABSTRACT

Disclosed are methods of treating a subject having high-risk multiple myeloma, methods of achieving negative minimal residual disease status in a subject having multiple myeloma, and methods of predicting a likelihood of, or decreasing a risk of, relapse and/or disease progression in a subject having multiple myeloma.

### RELATED APPLICATIONS

This application is a continuation application of U.S. application Ser. No. 18/175,345, filed Feb. 27, 2023, which is a divisional of U.S. application Ser. No. 16/177,239, filed Oct. 31, 2018, which issued as U.S. Pat. No. 11,618,787 on Apr. 4, 2023, which claims the benefit of U.S. Provisional Application No. 62/579,234, filed on Oct. 31, 2017, which applications are incorporated herein by reference in their entirety.

### INCORPORATION BY REFERENCE OF MATERIAL IN XML

This application incorporates by reference the Sequence Listing contained in the following eXtensible Markup Language (XML) file being submitted concurrently herewith:
a) File name: 01482024019.xml; created Apr. 29, 2026, 28,947 bytes in size.

### FIELD

Disclosed are methods of treating a subject having high-risk multiple myeloma, methods of achieving negative minimal residual disease status in a subject having multiple myeloma, and methods of predicting a likelihood of, or decreasing a risk of, relapse and/or disease progression in a subject having multiple myeloma.

### BACKGROUND

Multiple Myeloma (MM) is a B cell malignancy characterized by the latent accumulation of secretory plasma cells in bone marrow with a low proliferative index and an extended life span. The disease ultimately attacks bones and bone marrow, resulting in multiple tumors and lesions throughout the skeletal system. Approximately 1% of all cancers, and slightly more than 10% of all hematologic malignancies, can be attributed to MM. Incidence of MM increases in the aging population, with the median age at time of diagnosis being about 61 years.

Currently available therapies for MM include chemotherapy regimens, stem cell transplantation, THALOMID® (thalidomide), REVLIMID® (lenalidomide), POMALYST® (pomalidomide), VELCADE® (bortezomib), NINLARO® (ixazomib), KYPROLIS® (carfilzomib), FARADYK® (panobinostat), AREDIA® (pamidronate), ZOMETA® (zoledronic acid) and DARZALEX® (daratumumab). Current treatment protocols, which include a combination of chemotherapeutic agents such as vincristine, carmustine (BCNU), melphalan (Alkeran®), cyclophosphamide, doxorubicin (Adriamycin), and prednisone or dexamethasone, yield a complete remission rate of only about 5%, and median survival is approximately 36-48 months from the time of diagnosis. Recent advances using high dose chemotherapy followed by autologous bone marrow or peripheral blood mononuclear cell transplantation have increased the complete remission rate and remission duration. Nevertheless, overall survival has only been slightly prolonged, and no evidence for a cure has been obtained as yet. Ultimately, it is thought that all MM patients will relapse, even under maintenance therapy with interferon-alpha (IFN-α) alone or in combination with steroids.

Efficacy of the available drug treatment regimens for MM is limited by the low cell proliferation rate and development of drug resistance in up to 90% of patients. Chromosomal translocations, oncogene mutations, dysregulated signaling pathways such as anti-apoptotic and survival pathways, and bone marrow (BM) niche have been implicated to contribute to drug resistance in MM (for review, see Abdi et al., Oncotarget 4:2186-2207, 2013). The BM niche is implicated in proliferation, survival, differentiation, migration, and drug resistance of the malignant plasma cells (Manier et al., J Biomed Biotechnol 2012; published online 2012 Oct. 3, doi:_10.1155/_2012/_157496).

### SUMMARY

Disclosed herein are methods of treating a subject having high-risk multiple myeloma, comprising administering to the subject a therapeutically effective amount of an anti-CD38 antibody, a corticosteroid, and a non-corticosteroid chemotherapeutic agent for a time sufficient to treat the high-risk multiple myeloma.

Methods of achieving negative minimal residual disease status in a subject having multiple myeloma are also provided, the methods comprising administering to the subject a therapeutically effective amount of an anti-CD38 antibody, a corticosteroid, and a non-corticosteroid chemotherapeutic agent for a time sufficient to achieve negative minimal residual disease status.

Also provided are methods of predicting a likelihood of relapse and/or disease progression in a subject having multiple myeloma comprising measuring a minimal residual disease status in the subject, wherein the subject has received a therapeutically effective amount of an anti-CD38 antibody, a corticosteroid, and a non-corticosteroid chemotherapeutic agent, wherein a positive minimal residual disease status is indicative of a likelihood of relapse and/or disease progression.

Further disclosed are methods of decreasing a risk of relapse and/or disease progression in a subject having multiple myeloma comprising administering to the subject a therapeutically effective amount of an anti-CD38 antibody, a corticosteroid, and a non-corticosteroid chemotherapeutic agent to achieve a negative minimal residual disease status, wherein the negative residual disease status decreases the risk of relapse and/or disease progression.

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed. The drawings are not necessarily to scale, emphasis instead being placed upon illustrating embodiments. In the drawings:

FIG. 1 illustrates a graph showing percentage (%) of multiple myeloma subjects progression free and alive by cytogenetic risk status in the POLLUX (MMY3003) trial. DRd: daratumumab in combination with lenalinomide and dexamethasone; Rd: lenalinomide and dexamethasone. High Risk: subject has at least one of the following chromosomal abnormalities: t(4;14)(p16;q32); t(14;16)(q32;q23); or del17p. Standard Risk: subject has documented absence of any of the aforementioned chromosomal abnormalities.

FIG. 2 illustrates a graph showing percentage (%) of multiple myeloma subjects progression free and alive by cytogenetic risk status in the CASTOR (MMY3004) trial. DVd: daratumumab in combination with bortezomib and dexamethasone; Vd: bortezomib and dexamethasone. High Risk: subject has at least one of the following chromosomal abnormalities: t(4;14)(p16;q32); t(14;16)(q32;q23); or del17p. Standard Risk: subject has documented absence of any of the aforementioned chromosomal abnormalities.

FIG. 3 illustrates the number of sCR/CR patients that reached MRD negative status in POLLUX (MMY3003, top) and CASTOR (MMY3004, bottom) trials at indicated MRD negativity thresholds.

FIG. 4A illustrates a graph showing percentage (%) of multiple myeloma subjects progression free and alive over time (days) for the POLLUX (MMY3003) trial by MRD negativity at 10⁻⁴ threshold in DRd and Rd treatment arms.

FIG. 4B illustrates a graph showing percentage (%) of multiple myeloma subjects progression free and alive over time (days) for the POLLUX (MMY3003) trial by MRD negativity at 10⁻⁵ threshold in DRd and Rd treatment arms.

FIG. 4C illustrates a graph showing percentage (%) of multiple myeloma subjects progression free and alive over time (days) for the POLLUX (MMY3003) trial by MRD negativity at 10⁻⁶ threshold in DRd and Rd treatment arms.

FIG. 5A illustrates a graph showing percentage (%) of multiple myeloma subjects progression free and alive over time (days) for the CASTOR (MMY3004) trial by MRD negativity at 10⁻⁴ threshold in DVd and Vd treatment arms.

FIG. 5B illustrates a graph showing percentage (%) of multiple myeloma subjects progression free and alive over time (days) for the CASTOR (MMY3004) trial by MRD negativity at 10⁻⁵ threshold in DVd and Vd treatment arms.

FIG. 5C illustrates a graph showing percentage (%) of multiple myeloma subjects progression free and alive over time (days) for the CASTOR (MMY3004) trial by MRD negativity at 10⁻⁶ threshold in DVd and Vd treatment arms.

FIG. 6A illustrates a MRD profile of a responder who was MRD negative status (threshold 10⁻⁵) at suspected CR and remained MRD negative after CR. Malignant clone frequency at baseline (x=0) and over time is shown. The numbers vertically in black printed for each MRD sample show the MRD clone counts. The vertical dotted lines show the clinical response call for that subject, with the label printed at the bottom. Two distinct malignant clones (triangles and circles) were identified in the patient. The subject was MRD positive at baseline and MRD negative at and after suspected CR.

FIG. 6B illustrates a MRD profile of a responder who was MRD negative status (threshold 10⁻⁵) at suspected sCR and remained MRD negative after sCR. Malignant clone frequency at baseline (x=0) and over time is shown. The numbers vertically in black printed for each MRD sample show the MRD clone counts. The vertical dotted lines show the clinical response call for that subject, with the label printed at the bottom. Two distinct tumor clones (solid lines and dashed lines) were identified in the patient. The subject was MRD positive at baseline and MRD negative at and after suspected sCR.

FIG. 7A illustrates a MRD profile of a non-responder over time. Malignant clone frequency at baseline (x=0) and over time is shown. The vertical dotted lines show the clinical response call for that subject, with the label printed at the bottom. Two distinct tumor clones (solid lines and dashed lines) were identified in the patient. The subject was MRD positive at each evaluation point (threshold 10⁻⁵).

FIG. 7B illustrates a MRD profile of a non-responder over time. Malignant clone frequency at baseline (x=0) and over time is shown. The vertical dotted lines show the clinical response call for that subject, with the label printed at the bottom. Two distinct tumor clones (solid lines and dashed lines) were identified in the patient. The subject was MRD positive at each evaluation point (threshold 10⁻⁵).

FIG. 7C illustrates a MRD profile of a non-responder over time. Malignant clone frequency at baseline (x=0) and over time is shown. The vertical dotted lines show the clinical response call for that subject, with the label printed at the bottom. Two distinct tumor clones (solid lines and dashed lines) were identified in the patient. The subject was MRD positive at each evaluation point (threshold 10⁻⁵).

FIG. 7D illustrates a MRD profile of a non-responder over time. Malignant clone frequency at baseline (x=0) and over time is shown. The vertical dotted lines show the clinical response call for that subject, with the label printed at the bottom. Two distinct tumor clones (solid lines and dashed lines) were identified in the patient. The subject was MRD positive at each evaluation point (threshold 10⁻⁵).

FIG. 8A illustrates a MRD profile of a subject who initially showed clinical response after which the subject experienced progressive disease. Malignant clone frequency at baseline (x=0) and over time is shown. The vertical dotted lines show the clinical response call for that subject, with the label printed at the bottom. Three distinct tumor clones (solid lines, dashed lines and dotted lines) were identified in the patient. The subject was MRD positive at each evaluation point (threshold 10⁻⁵).

FIG. 8B illustrates a MRD profile of a subject who initially showed clinical response after which the subject experienced progressive disease. Malignant clone frequency at baseline (x=0) and over time is shown. The vertical dotted lines show the clinical response call for that subject, with the label printed at the bottom. Two distinct tumor clones (solid lines and dashed lines) were identified in the patient. The subject was MRD positive at each evaluation point (threshold 10⁻⁵).

FIG. 9A illustrates aMRD profile of a subject who showed rapid clinical response but reached MRD negativity only after suspected CR. Malignant clone frequency at baseline (x=0) and over time is shown. The vertical dotted lines show the clinical response call for that subject, with the label printed at the bottom. Two distinct tumor clones (solid lines and dashed lines) were identified in the patient. The subject was MRD positive at baseline and at suspected CR, and MRD negative at approximately 340 days after initiation of the treatment (threshold 10⁻⁵).

FIG. 9B illustrates a MRD profile of a subject who showed rapid clinical response but reached MRD negativity only after suspected CR. Malignant clone frequency at baseline (x=0) and over time is shown. The vertical dotted lines show the clinical response call for that subject, with the label printed at the bottom. Two distinct tumor clones (solid lines and dashed lines) were identified in the patient. The subject was MRD positive at baseline, at suspected CR, at approximately 170 days and 260 days after initiation of the treatment, and MRD negative at approximately 360 days after initiation of the treatment (threshold 10⁻⁵).

FIG. 10A illustrates a MRD profile of a subject who showed slow clinical response and remained MRD positive after suspected CR. Malignant clone frequency at baseline (x=0) and over time is shown. The vertical dotted lines show the clinical response call for that subject, with the label printed at the bottom. Two distinct tumor clones (solid lines and dashed lines) were identified in the patient. The subject was MRD positive at each evaluation point (threshold 10⁻⁵).

FIG. 10B illustrates a MRD profile of a subject who showed slow clinical response and remained MRD positive after suspected CR. Malignant clone frequency at baseline (x=0) and over time is shown. The vertical dotted lines show the clinical response call for that subject, with the label printed at the bottom. Two distinct tumor clones (solid lines and dashed lines) were identified in the patient. The subject was MRD positive at each evaluation point (threshold 10⁻⁵).

FIG. 10C illustrates a MRD profile of a subject who showed slow clinical response and remained MRD positive after suspected CR. Malignant clone frequency at baseline (x=0) and over time is shown. The vertical dotted lines show the clinical response call for that subject, with the label printed at the bottom. Two distinct tumor clones (solid lines and dashed lines) were identified in the patient. The subject was MRD positive at each evaluation point (threshold 10⁻⁵).

FIG. 10D illustrates a MRD profile of a subject who showed slow clinical response and remained MRD positive after suspected CR. Malignant clone frequency at baseline (x=0) and over time is shown. The vertical dotted lines show the clinical response call for that subject, with the label printed at the bottom. Two distinct tumor clones (solid lines and dashed lines) were identified in the patient. The subject was MRD positive at each evaluation point (threshold 10⁻⁵).

FIG. 11A illustrates the percentage (%) of MRD-negative subjects at indicated MRD negativity thresholds (10⁻⁴, 10⁻⁵ or 10⁻⁶) in standard-risk patients from the POLLUX (MMY3003) trial. Light bars: patients that received lenalinomide and dexamethasone alone (Rd); Dark bars: patients that received daratumumab, lenalinomide, and dexamethasone (DRd). **p<0.005; ***p<0.0001 between indicated DRd vs Rd subject groups.

FIG. 11B illustrates the percentage (%) of MRD-negative subjects at indicated MRD negativity thresholds (10⁻⁴, 10⁻⁵ or 10⁻⁶) in high-risk patients from the POLLUX (MMY3003) trial. Light bars: patients that received lenalinomide and dexamethasone alone (Rd); Dark bars: patients that received daratumumab, lenalinomide, and dexamethasone (DRd). *p<0.05 between indicated DRd vs Rd subject groups.

FIG. 12A illustrates the percentage (%) of MRD-negative subjects at indicated MRD negativity thresholds (10⁻⁴, 10⁻⁵ or 10⁻⁶) in standard-risk patients from the CASTOR (MMY3004) trial. Light bars: patients that received bortezomib and dexamethasone (Vd); Dark bars: patients that received daratumumab, bortezomib, and dexamethasone (DVd). *p<0.05; **p<0.005 between indicated DVd vs Vd subject groups.

FIG. 12B illustrates the percentage (%) of MRD-negative subjects at indicated MRD negativity thresholds (10⁻⁴, 10⁻⁵ or 10⁻⁶) in high-risk patients from the CASTOR (MMY3004) trial. Light bars: patients that received bortezomib and dexamethasone (Vd); Dark bars: patients that received daratumumab, bortezomib, and dexamethasone (DVd). *p<0.05 in DVd vs Vd subject groups.

### DETAILED DESCRIPTION

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of 0.5% or less, or variations of ±0.1% or less from the specified value.

The term “comprising” is intended to include examples encompassed by the terms “consisting essentially of” and “consisting of”; similarly, the term “consisting essentially of” is intended to include examples encompassed by the term “consisting of.”

“CD38” refers to the human CD38 protein (UniProtKB accession no. P28907) (synonyms: ADP-ribosyl cyclase 1, cADPr hydrolase 1, cyclic ADP-ribose hydrolase 1). Human CD38 has an amino acid sequence as shown in SEQ ID NO: 1. CD38 is a single pass type II transmembrane protein with amino acid residues 1-21 representing the cytosolic domain, amino acid residues 22-42 representing the transmembrane domain, and residues 43-300 representing the extracellular domain. Anti-CD38 antibodies are described, for example, in Int'l Pat. Pub. No. WO2008/037257, Int'l Pat. Pub. No. WO2008/047242 and Int'l Pat. Pub. No. WO2007/042309.

The term “antibody,” and like terms is meant in a broad sense and includes immunoglobulin molecules including, monoclonal antibodies (such as murine, human, human-adapted, humanized, and chimeric monoclonal antibodies), antibody fragments, bispecific or multispecific antibodies, dimeric, tetrameric or multimeric antibodies, and single chain antibodies.

Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (λ), based on the amino acid sequences of their constant domains.

“Antibody fragment” refers to a portion of an immunoglobulin molecule that retains the antigen binding properties of the parental full length antibody. Exemplary antibody fragments are heavy chain complementarity determining regions (HCDR) 1, 2, and/or 3, light chain complementarity determining regions (LCDR) 1, 2, and/or 3, a heavy chain variable region (VH), or a light chain variable region (VL). Antibody fragments include: a Fab fragment, a monovalent fragment consisting of the VL, VH, constant light (CL), and (constant heavy 1) CH1 domains; a F(ab)₂ fragment, a bivalent fragment comprising two Fab fragments linked by a disulfide bridge at the hinge region; a Fd fragment consisting of the VH and CHI domains; a Fv fragment consisting of the VL and VH domains of a single arm of an antibody; and a domain antibody (dAb) fragment (Ward et al., Nature 341:544-546, 1989), which consists of a VH domain or a VL domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in Int'l Pat. Pub. Nos. WO1998/44001, WO1988/01649, WO1994/13804, and WO1992/01047. These antibody fragments are obtained using techniques well known to those of skill in the art, and the fragments are screened for utility in the same manner as are full length antibodies.

The phrase “isolated antibody” refers to an antibody or antibody fragment that is substantially free of other antibodies having different antigenic specificities (e.g., an isolated anti-CD38 antibody is substantially free of antibodies that specifically bind antigens other than human CD38). An isolated anti-CD38 antibody, however, can have cross-reactivity to other antigens, such as orthologs of human CD38, such as Macaca fascicularis (cynomolgus) CD38. Moreover, an isolated antibody may be substantially free of other cellular material and/or chemicals.

An antibody variable region consists of a “framework” region interrupted by three “antigen binding sites.” The antigen binding sites are defined using various terms: (i) Complementarity Determining Regions (CDRs), three in the VH (HCDR1, HCDR2, HCDR3), and three in the VL (LCDR1, LCDR2, LCDR3) are based on sequence variability (Wu and Kabat, J Exp Med 132:211-50, 1970; Kabat et al. Sequences of Proteins of Immunological Interest, 5th Ed. Public Health Service, National Institutes of Health, Bethesda, Md., 1991); and (ii) “Hypervariable regions” (“HVR” or “HV”), three in the VH (H1, H2, H3) and three in the VL (L1, L2, L3) refer to the regions of the antibody variable domains which are hypervariable in structure as defined by Chothia and Lesk (Chothia and Lesk, Mol Biol 196:901-17, 1987). Other terms include “IMGT-CDRs” (Lefranc et al., Dev Comparat Immunol 27:55-77, 2003) and “Specificity Determining Residue Usage” (SDRU) (Almagro, Mol Recognit 17:132-43, 2004). The International ImMunoGeneTics (IMGT) database (www_imgt_org) provides a standardized numbering and definition of antigen-binding sites. The correspondence between CDRs, HVs and IMGT delineations is described in Lefranc et al., Dev Comparat Immunol 27:55-77, 2003.

“Framework” or “framework sequences” are the remaining sequences of a variable region other than those defined to be antigen binding sites. Because the antigen binding sites can be defined by various terms as described above, the exact amino acid sequence of a framework depends on how the antigen-binding site was defined.

“Humanized antibody” refers to an antibody in which the antigen binding sites are derived from non-human species and the framework regions are derived from human immunoglobulin sequences. Humanized antibodies may include substitutions in the framework regions so that the framework may not be an exact copy of expressed human immunoglobulin or germline gene sequences. If the antibody contains a constant region, the constant region is also derived from sequences of human origin. “Derived from,” as used in the context of humanized antibodies, means that the region in question is at least 80% homologous in sequence to the corresponding region of the immunoglobulin from the species in which it is based.

“Human-adapted” antibodies or “human framework adapted (HFA)” antibodies refers to humanized antibodies adapted according to methods described in U.S. Pat. Publ. No. US2009/0118127. Human-adapted antibodies are humanized by selecting the acceptor human frameworks based on the maximum CDR and FR similarities, length compatibilities and sequence similarities of CDR1 and CDR2 loops and a portion of light chain CDR3 loops.

“Human antibody” refers to an antibody having heavy and light chain variable regions in which both the framework and the antigen binding sites are derived from sequences of human origin. If the antibody contains a constant region, the constant region also is derived from sequences of human origin. A human antibody comprises heavy or light chain variable regions that are “derived from” sequences of human origin if the variable regions of the antibody are obtained from a system that uses human germline immunoglobulin or rearranged immunoglobulin genes. Such systems include human immunoglobulin gene libraries displayed on phage, and transgenic non-human animals such as mice carrying human immunoglobulin loci as described herein. “Human antibody” may contain amino acid differences when compared to the human germline or rearranged immunoglobulin sequences due to, for example, naturally occurring somatic mutations or intentional introduction of substitutions in the framework or antigen binding sites. Typically, a “human antibody” is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical in amino acid sequence to an amino acid sequence encoded by a human germline or rearranged immunoglobulin gene. In some cases, a “human antibody” may contain consensus framework sequences derived from human framework sequence analyses, for example as described in Knappik et al., J Mol Biol 296:57-86, 2000, or synthetic HCDR3 incorporated into human immunoglobulin gene libraries displayed on phage, as described in, for example, Shi et al., J Mol Biol 397:385-96, 2010 and Int'l Pat. Pub. No. WO2009/085462. Antibodies in which antigen binding sites are derived from a non-human species are not included in the definition of “human antibody”.

Isolated humanized antibodies may be synthetic. Human antibodies, while derived from human immunoglobulin sequences, may be generated using systems such as phage display incorporating synthetic CDRs and/or synthetic frameworks, or can be subjected to in vitro mutagenesis to improve antibody properties, resulting in antibodies that do not naturally exist within the human antibody germline repertoire in vivo.

“Recombinant antibody” includes all antibodies that are prepared, expressed, created, or isolated by recombinant means, such as: antibodies isolated from an animal (e.g., a mouse) that is transgenic or transchromosomal for human immunoglobulin genes or a hybridoma prepared therefrom (described further below); antibodies isolated from a host cell transformed to express the antibody; antibodies isolated from a recombinant, combinatorial antibody library; and antibodies prepared, expressed, created, or isolated by any other means that involve splicing of human immunoglobulin gene sequences to other DNA sequences, or antibodies that are generated in vitro using Fab arm exchange.

“Monoclonal antibody” refers to a preparation of antibody molecules of a single molecular composition. A monoclonal antibody composition displays a single binding specificity and affinity for a particular epitope, or in a case of a bispecific monoclonal antibody, a dual binding specificity to two distinct epitopes. Monoclonal antibody therefore refers to an antibody population with single amino acid composition in each heavy and each light chain, except for possible well-known alterations such as removal of C-terminal lysine from the antibody heavy chain. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Epitope” refers to a portion of an antigen to which an antibody specifically binds. Epitopes usually consist of chemically active (such as polar, non-polar, or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Variant” refers to a polypeptide or a polynucleotide that differs from a reference polypeptide or a reference polynucleotide by one or more modifications for example, substitutions, insertions, or deletions.

“In combination with” means that two or more therapeutics can be administered to a subject together in a mixture, concurrently as single agents, or sequentially as single agents in any order.

“Treat,” “treatment,” and like terms refer to both therapeutic treatment and prophylactic or preventative measures, and includes reducing the severity and/or frequency of symptoms, eliminating symptoms and/or the underlying cause of the symptoms, reducing the frequency or likelihood of symptoms and/or their underlying cause, improving or remediating damage caused, directly or indirectly, by multiple myeloma. Treatment also includes prolonging survival as compared to the expected survival of a subject not receiving treatment. Subjects to be treated include those that have the condition or disorder as well as those prone to have the condition or disorder or those in which the condition or disorder is to be prevented.

“Therapeutically effective amount” refers to an amount of the disclosed combination therapy effective, at dosages and for periods of time necessary, to achieve a desired treatment. A therapeutically effective amount may vary according to factors such as the disease state, age, sex, and weight of the subject, and the ability of the combination therapy to elicit a desired response in the subject. Exemplary indicators of a therapeutically effect amount include, for example, improved well-being of the patient, reduction of a tumor burden, arrested or slowed growth of a tumor, and/or absence of metastasis of cancer cells to other locations in the body.

“Inhibit growth” (e.g., referring to cells, such as tumor cells) refers to a measurable decrease in in vitro or in vivo cell growth upon contact with the combination therapy when compared to the growth of the same cells in the absence of the combination therapy. Inhibition of growth of a cell in vitro or in vivo may be at least about 10%, about 20%, about 30%, about 40%, about 50%, about 60%, about 70%, about 80%, about 90%, about 99%, or about 100%. Inhibition of cell growth can occur by a variety of mechanisms, for example by antibody-mediated ADCC, ADCP and/or CDC, apoptosis, necrosis, or by inhibition of cell proliferation.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

The following abbreviations are used throughout the disclosure: bone marrow aspirates (BMA); complete response (CR); daratumumab, bortezomib, and dexamethasone (DVd); daratumumab, lenalinomide, and dexamethasone (DRd); International Myeloma Working Group (IMWG); International Staging System (ISS); minimal residual disease (MHRD); multiple myeloma (MM); partial response (PR); progression-free survival (PFS); overall response rate (ORR); overall survival (OS); lenalinomide and dexamethasone (Rd); stringent complete response (sCR); time to disease progression (TTP); bortezomib and dexamethasone (Vd); very good partial response (VGPR); antibody-dependent cellular cytotoxicity (ADCC), antibody-dependent cellular phagocytosis (ADCP), bortezomib (Bort); complement-dependent cytotoxicity (CDC), complementarity determining region (CDR), constant light (CL), (constant heavy 1) CH1 domains, daratumumab (DARA); heavy chain CDR (HCDR), heavy chain variable region (VH), lenalidomide (LEN); light chain CDR (LCDR), light chain variable region (VL); patients (pts).

### Methods of Treating a Subject Having High-Risk Multiple-Myeloma

Disclosed herein are methods of treating a subject having high-risk multiple myeloma, comprising administering to the subject a therapeutically effective amount of an anti-CD38 antibody, a corticosteroid, and a non-corticosteroid chemotherapeutic agent for a time sufficient to treat the high-risk multiple myeloma.

Any anti-CD38 antibody may be used in the disclosed methods. For example, the variable regions of the anti-CD38 antibodies may be obtained from existing anti-CD38 antibodies and optionally cloned as full length antibodies using standard methods. Exemplary antibody variable regions that bind CD38 that may be used are described in Int'l Pat. Pub. Nos. WO2005/103083, WO2006/125640, WO2007/042309, WO2008/047242, WO2012/092612, WO2006/099875, and WO2011/154453A1.

The anti-CD38 antibody can bind to a region of human CD38 comprising SKRNIQFSCKNIYR (SEQ ID NO: 2) and a region of human CD38 comprising EKVQTLEAWVIHGG (SEQ ID NO: 3). An anti-CD38 antibody binds to a region of human CD38 comprising SEQ ID NO: 2 and a region of human CD38 comprising SEQ ID NO: 3 when the antibody binds at least 1, 2, 3, 4, 5, 6, 7, 8, 9, 10, 11, 12, 13, or 14 residues within SEQ ID NO: 2 and at least 1, 2, 3, 4, 5, 6, 7, 8, 9, 10, 11, 12, 13, or 14 residues within SEQ ID NO: 3. In some embodiments, the anti-CD38 antibody binds at least one amino acid in a region of human CD38 comprising SEQ ID NO: 2 and at least one amino acid in a region of human CD38 comprising SEQ ID NO: 3. In some embodiments, the anti-CD38 antibody binds at least two amino acids in a region of human CD38 comprising SEQ ID NO: 2 and at least two amino acids in a region of human CD38 comprising SEQ ID NO: 3. In some embodiments, the anti-CD38 antibody binds at least three amino acids in a region of human CD38 comprising SEQ ID NO: 2 and at least three amino acids in a region of human CD38 comprising SEQ ID NO: 3. Antibodies binding to a region of human CD38 comprising SEQ ID NO: 2 and a region of human CD38 comprising SEQ ID NO: 3 may be generated, for example, by immunizing mice with peptides having amino acid sequences comprising SEQ ID NOs: 2 and 3 using standard methods and as described herein, and characterizing the obtained antibodies for binding to the peptides using, for example, ELISA or mutagenesis studies.

An exemplary anti-CD38 antibody that binds to a region of human CD38 comprising SEQ ID NO: 2 and a region of human CD38 comprising SEQ ID NO: 3 is DARZALEX™ (daratumumab), which comprises:
a heavy chain amino acid sequence of SEQ ID NO: 12 and light chain amino acid sequence of SEQ ID NO: 13;
a heavy chain variable region (VH) of SEQ ID NO: 4 and a light chain variable region (VL) of SEQ ID NO: 5; and
a heavy chain complementarity determining region (CDR) 1, a heavy chain CDR2, and a heavy chain CDR3 of SEQ ID NOs: 6, 7, and 8, respectively, and a light chain complementarity determining region (CDR) 1, a light chain CDR2, and a light chain CDR3 of SEQ ID NOs: 9, 10, and 11, respectively.

The anti-CD38 antibody can comprise a heavy chain CDR1 comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 8, a light chain CDR1 comprising the amino acid sequence of SEQ ID NO: 9, a light chain CDR2 comprising the amino acid sequence of SEQ ID NO: 10, and a light chain CDR3 comprising the amino acid sequence of SEQ ID NO: 11. The anti-CD38 antibody can comprise a VH comprising an amino acid sequence that is 95%, 96%, 97%, 98%, 99%, or 100% identical to that of SEQ ID NO: 4 and a VL comprising an amino acid sequence that is 95%, 96%, 97%, 98%, 99%, or 100% identical to that of SEQ ID NO: 5. In some embodiments, the anti-CD38 antibody can comprise a VH comprising the amino acid sequence of SEQ ID NO: 4 and a VL comprising the amino acid sequence of SEQ ID NO: 5. The anti-CD38 antibody can comprise a heavy chain comprising the amino acid sequence of SEQ ID NO: 12 and a light chain comprising the amino acid sequence of SEQ ID NO: 13.

The anti-CD38 antibody can comprise a heavy chain CDR1, a heavy chain CDR2, and a heavy chain CDR3 of a VH comprising the amino acid sequence of SEQ ID NO: 14, and a light chain CDR1, a light chain CDR2, and a light chain CDR3 of a VL comprising the amino acid sequence of SEQ ID NO:15. The anti-CD38 antibody can comprise a VH comprising an amino acid sequence that is 95%, 96%, 97%, 98%, 99%, or 100% identical to that of SEQ ID NO: 15 and a VL comprising an amino acid sequence that is 95%, 96%, 97%, 98%, 99%, or 100% identical to that of SEQ ID NO: 15. In some embodiments, the anti-CD38 antibody can comprise a VH comprising the amino acid sequence of SEQ ID NO: 14 and a VL comprising the amino acid sequence of SEQ ID NOs:15. In some embodiments, for example, the anti-CD38 antibody can comprise mAb003 (described in U.S. Pat. No. 7,829,693, incorporated herein by reference).

The anti-CD38 antibody can comprise a heavy chain CDR1, a heavy chain CDR2, and a heavy chain CDR3 of a VH comprising the amino acid sequence of SEQ ID NO: 16, and a light chain CDR1, a light chain CDR2, and a light chain CDR3 of a VL comprising the amino acid sequence of SEQ ID NO: 17. The anti-CD38 antibody can comprise a VH comprising an amino acid sequence that is 95%, 96%, 97%, 98%, 99%, or 100% identical to that of SEQ ID NO: 16 and a VL comprising an amino acid sequence that is 95%, 96%, 97%, 98%, 99%, or 100% identical to that of SEQ ID NO: 17. In some embodiments, the anti-CD38 antibody can comprise a VH comprising the amino acid sequence of SEQ ID NO: 16 and a VL comprising the amino acid sequence of SEQ ID NO: 17. In some embodiments, for example, the anti-CD38 antibody can comprise mAb024 (described in U.S. Pat. No. 7,829,693, incorporated herein by reference).

The anti-CD38 antibody can comprise a heavy chain CDR1, a heavy chain CDR2, and a heavy chain CDR3 of a VH comprising the amino acid sequence of SEQ ID NO: 18, and a light chain CDR1, a light chain CDR2, and a light chain CDR3 of a VL comprising the amino acid sequence of SEQ ID NO: 19. The anti-CD38 antibody can comprise a VH comprising an amino acid sequence that is 95%, 96%, 97%, 98%, 99%, or 100% identical to that of SEQ ID NO: 18 and a VL comprising an amino acid sequence that is 95%, 96%, 97%, 98%, 99%, or 100% identical to that of SEQ ID NO: 19. In some embodiments, the anti-CD38 antibody can comprise a VH comprising the amino acid sequence of SEQ ID NO: 18 and a VL comprising the amino acid sequence of SEQ ID NO: 19. In some embodiments, for example, the anti-CD38 antibody can comprise MOR-202 (MOR-03087) (described in U.S. Pat. No. 8,088,896, incorporated herein by reference).

The anti-CD38 antibody can comprise a heavy chain CDR1, a heavy chain CDR2, and a heavy chain CDR3 of a VH comprising the amino acid sequence of SEQ ID NO: 20, and a light chain CDR1, a light chain CDR2, and a light chain CDR3 of a VL comprising the amino acid sequence of SEQ ID NO: 21. The anti-CD38 antibody can comprise a VH comprising an amino acid sequence that is 95%, 96%, 97%, 98%, 99%, or 100% identical to that of SEQ ID NO: 20 and a VL comprising an amino acid sequence that is 95%, 96%, 97%, 98%, 99%, or 100% identical to that of SEQ ID NO: 21. In some embodiments, the anti-CD38 antibody can comprise a VH comprising the amino acid sequence of SEQ ID NO: 20 and a VL comprising the amino acid sequence of SEQ ID NO: 21. In some embodiments, for example, the anti-CD38 antibody can comprise isatuximab (described in U.S. Pat. No. 8,153,765, incorporated herein by reference). In some aspects, the VH and the VL of isatuximab may be expressed as IgG1/K.

Antibodies that are substantially identical to those disclosed herein may be used in the disclosed methods. The term “substantially identical” means that the antibody heavy chain or light chain amino acid sequences are identical, or have “insubstantial differences,” compared to the antibody disclosed herein. Insubstantial differences are substitutions of 1, 2, 3, 4, 5, 6, 7, 8, 9, 10, 11, 12, 13, 14, or 15 amino acids in an antibody heavy chain or light chain that do not adversely affect antibody properties. Antibody sequences can be compared, for example, by pairwise alignments using the default settings of the AlignX module of Vector NTI v.9.0.0 (Invitrogen, Carlsbad, CA). The protein sequences of the disclosed antibodies can be used as a query sequence to perform a search against public or patent databases to, for example, identify related sequences. Exemplary programs used to perform such searches are the XBLAST or BLASTP programs (www_ncbi_nlm/nih_gov), or the GenomeQuest™ (GenomeQuest, Westborough, MA) suite using the default settings. Antibodies that are substantially identical to the disclosed antibodies can be generated, for example, by making conservative modifications to the amino acid sequences of the disclosed antibodies. “Conservative modifications” refer to amino acid modifications that do not significantly affect or alter the binding characteristics of the antibody containing the amino acid sequences. Conservative modifications include amino acid substitutions, additions, and deletions. “Conservative substitutions” are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), an amide (e.g., asparagine, glutamine), beta-branched side chains (e.g., threonine, valine, isoleucine), and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643:55-67; Sasaki et al., (1988) Adv Biophys 35:1-24). Exemplary substitutions that can be made to the anti-CD38 antibodies used in the disclosed methods include, for example, conservative substitutions with an amino acid having similar charge, hydrophobic, or stereochemical characteristics. Conservative substitutions may also be made to improve antibody properties, including stability or affinity, or to improve antibody effector functions. 1, 2, 3, 4, 5, 6, 7, 8, 9, 10, 11, 12, 13, 14, or 15 amino acid substitutions may be made, for example, to the heavy and/or the light chain of the anti-CD38 antibody. Furthermore, any native residue in the heavy and/or light chain may also be substituted with alanine, as has been previously described for alanine scanning mutagenesis (MacLennan et al., Acta Physiol Scand Suppl 643:55-67, 1998; Sasaki et al., Adv Biophys 35:1-24, 1998). Suitable amino acid substitutions may be determined by those skilled in the art at the time such substitutions are desired. Amino acid substitutions may be performed, for example, by PCR mutagenesis (as disclosed in U.S. Pat. No. 4,683,195). Libraries of variants may be generated using well-known methods; for example using random (NNK) or non-random codons (for example DVK codons) which encode 11 amino acids (Ala, Cys, Asp, Glu, Gly, Lys, Asn, Arg, Ser, Tyr, Trp) and screening the libraries for variants with desired properties. The generated variants may be tested for their binding to CD38 and their ability to induce ADCC using methods described herein.

The anti-CD38 antibody can be of the IgG1, IgG2, IgG3, or IgG4 isotype. In some embodiments, the anti-CD38 antibody is of the IgG1 isotype. In some embodiments, the anti-CD38 antibody is of the IgG2 isotype. In some embodiments, the anti-CD38 antibody is of the IgG3 isotype. In some embodiments, the anti-CD38 antibody is of the IgG4 isotype.

Anti-CD38 antibodies used in the disclosed methods may also be selected de novo from, for example, a phage display library, where the phage is engineered to express human immunoglobulins or portions thereof such as Fabs, single chain antibodies (scFv), or unpaired or paired antibody variable regions (Knappik et al., J Mol Biol 296:57-86, 2000; Krebs et al., J Immunol Meth 254:67-84, 2001; Vaughan et al., Nature Biotechnology 14:309-314, 1996; Sheets et al., PITAS (USA) 95:6157-6162, 1998; Hoogenboom and Winter, J Mol Biol 227:381, 1991; Marks et al., J Mol Biol 222:581, 1991). CD38 binding variable domains may be isolated, for example, from phage display libraries expressing antibody heavy and light chain variable regions as fusion proteins with bacteriophage pIX coat protein as described in Shi et al (2010) J. Mol. Biol. 397:385-96 and Int'l Pat. Pub. No. WO2009/085462. The antibody libraries may be screened for binding to human CD38 extracellular domain and the obtained positive clones may be further characterized and the Fabs isolated from the clone lysates, and subsequently cloned as full length antibodies. Such phage display methods for isolating human antibodies are established in the art. See for example: U.S. Pat. Nos. 5,223,409; 5,403,484; 5,571,698; 5,427,908; 5,580,717; 5,969,108; 6,172,197; 5,885,793; 6,521,404; 6,544,731; 6,555,313; 6,582,915; and 6,593,081.

In some embodiments, the anti-CD38 antibody competes for binding to CD38 with a reference antibody comprising:
a) a heavy chain CDR1 comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 8, a light chain CDR1 comprising the amino acid sequence of SEQ ID NO: 9, a light chain CDR2 comprising the amino acid sequence of SEQ ID NO: 10, and a light chain CDR3 comprising an amino acid sequence of SEQ ID NO: 11;
b) a VH comprising the amino acid sequence of SEQ ID NO: 4 and a VL comprising the amino acid sequence of SEQ ID NO: 5;
c) a heavy chain comprising the amino acid sequence of SEQ ID NO: 12 and a light chain comprising the amino acid sequence of SEQ ID NO: 13;
d) DARZALEX™ (daratumumab);
e) a heavy chain CDR1, a heavy chain CDR2, and a heavy chain CDR3 of a VH comprising the amino acid sequence of SEQ ID NO: 14, and a light chain CDR1, a light chain CDR2, and a light chain CDR3 of a VL comprising the amino acid sequence of SEQ ID NO: 15;
f) a VH comprising the amino acid sequence of SEQ ID NO: 14 and a VL comprising the amino acid sequence of SEQ ID NO: 15;
g) mAb003;
h) a heavy chain CDR1, a heavy chain CDR2, and a heavy chain CDR3 of a VH comprising the amino acid sequence of SEQ ID NO: 16, and a light chain CDR1, a light chain CDR2, and a light chain CDR3 of a VL comprising the amino acid sequence of SEQ ID NO: 17;
i) a VH comprising the amino acid sequence of SEQ ID NO: 16 and a VL comprising the amino acid sequence of SEQ ID NO: 17;
j) mAb024;
k) a heavy chain CDR1, a heavy chain CDR2, and a heavy chain CDR3 of a VH comprising the amino acid sequence of SEQ ID NO: 18, and a light chain CDR1, a light chain CDR2, and a light chain CDR3 of a VL comprising the amino acid sequence of SEQ ID NO: 19;
l) a VH comprising the amino acid sequence of SEQ ID NO: 18 and a VL comprising the amino acid sequence of SEQ ID NO: 19;
m) MOR-202 (MOR-03087);
n) a heavy chain CDR1, a heavy chain CDR2, and a heavy chain CDR3 of a VH comprising the amino acid sequence of SEQ ID NO: 20, and a light chain CDR1, a light chain CDR2, and a light chain CDR3 of a VL comprising the amino acid sequence of SEQ ID NO: 21;
o) a VH comprising the amino acid sequence of SEQ ID NO: 20 and a VL comprising the amino acid sequence of SEQ ID NO: 21;
p) isatuximab; or
q) any combination of a) to p).

Antibodies may be evaluated for their competition with a reference antibody (such as references antibodies a) to q) above) for binding to CD38 using well known in vitro methods. In an exemplary method, CHO cells recombinantly expressing CD38 may be incubated with unlabeled reference antibody for 15 min at 4° C., followed by incubation with an excess of fluorescently labeled test antibody for 45 min at 4° C. After washing in PBS/BSA, fluorescence may be measured by flow cytometry using standard methods. In another exemplary method, an extracellular portion of human CD38 may be coated on the surface of an ELISA plate. Excess unlabeled reference antibody may be added for about 15 minutes and subsequently biotinylated test antibodies may be added. After washes in PBS/Tween, binding of the test biotinylated antibody may be detected using horseradish peroxidase (HRP)-conjugated streptavidin and the signal detected using standard methods. In the competition assays, the reference antibody may be labeled and the test antibody may be unlabeled. The test antibody competes with the reference antibody when the reference antibody inhibits binding of the test antibody, or the test antibody inhibits binding of the reference antibody, by at least about 90%, 95%, or 100%. The epitope of the test antibody may further be defined, for example, by peptide mapping or hydrogen/deuterium protection assays using known methods, or by crystal structure determination.

The anti-CD38 antibody can induce killing of CD38-expressing cells by antibody-dependent cellular cytotoxicity (ADCC), antibody-dependent cellular phagocytosis (ADCP), complement-dependent cytotoxicity (CDC), or apoptosis. In some embodiments, the anti-CD38 antibody induces killing of CD38-expressing cells by ADCC. In some embodiments, the anti-CD38 antibody induces killing of CD38-expressing cells by ADCP. In some embodiments, the anti-CD38 antibody induces killing of CD38-expressing cells by CDC. In some embodiments, the anti-CD38 antibody induces killing of CD38-expressing cells by apoptosis. In some embodiments, the anti-CD38 antibody induces killing of CD38-expressing cells by any combination of ADCC, ADCP, CDC, and apoptosis.

“Antibody-dependent cellular cytotoxicity,” “antibody-dependent cell-mediated cytotoxicity” or “ADCC” is a mechanism for inducing cell death that depends upon the interaction of antibody-coated target cells with effector cells possessing lytic activity, such as natural killer (NK) cells, monocytes, macrophages, and neutrophils via Fc gamma receptors (FcγR) expressed on effector cells. For example, NK cells express FcγRIIIa, whereas monocytes express FcγRI, FcγRII, and FcγRIIIa. Death of the antibody-coated target cell, such as CD38-expressing MM cell, occurs as a result of effector cell activity through the secretion of membrane pore-forming proteins and proteases. To assess ADCC activity of an anti-CD38 antibody, the antibody may be added to CD38-expressing cells in combination with immune effector cells, which may be activated by the antigen/antibody complexes resulting in cytolysis of the target cell. Cytolysis may be detected by the release of a label (e.g., radioactive substrates, fluorescent dyes, or natural intracellular proteins) from the lysed cells. Exemplary effector cells for such assays include peripheral blood mononuclear cells (PBMC) and NK cells. Multiple myeloma cell lines or primary MM cells that express CD38 may be used as target cells. In an exemplary assay, MM cell lines engineered to express luciferase are incubated with anti-CD38 antibodies. Freshly isolated PBMC effector cells are added at a target:effector cell ratio of 40:1. 4 hours after addition of PBMC, luciferin is added and the resulting bioluminescent signal emitted from surviving MM cells can be determined within 20 minutes using a luminometer (SpectraMax, Molecular Devices), and the percentage ADCC of MM cells can calculated using the formula: % ADCC=1−(mean bioluminescent signal in the absence of PBMCs/mean bioluminescent signal in the presence of PBMCs)×100%. Anti-CD38 antibodies used in the disclosed methods can induce ADCC by about 20%, 25%, 30%, 35%, 40%, 45%, 50%, %55%, 60%, 65%, 70%, 80%, 90%, or 100%.

“Complement-dependent cytotoxicity,” or “CDC,” refers to a mechanism for inducing cell death in which an Fc effector domain of a target-bound antibody binds and activates complement component C1q, which in turn activates the complement cascade leading to target cell death. Activation of complement may also result in deposition of complement components on the target cell surface that facilitate ADCC by binding complement receptors (e.g., CR3) on leukocytes. In an exemplary assay, primary BM-MNC cells isolated from a patient with a B-cell malignancy may be treated with an anti-CD38 antibody and complement derived from 10% pooled human serum for 1 hour at a concentration of 0.3-10 μg/ml, and the survival of primary CD38⁺ MM cells may be determined by flow cytometry using techniques described in van der Veer et al., Haematologica 96:284-290, 2011; van der Veer et al., Blood Cancer J 1(10):e41, 2011. The percentage of MM cell lysis may be determined relative to an isotype control as described herein. Anti-CD38 antibodies used in the disclosed methods may induce CDC by about 20%, 25%, 30%, 35%, 40%, 45%, 50%, 55%, 60%, 65%, 70%, 75%, 80%, 85%, 90%, 95%, or 100%.

“Antibody-dependent cellular phagocytosis,” or “ADCP,” refers to a mechanism of elimination of antibody-coated target cells by internalization by phagocytic cells, such as macrophages or dendritic cells. ADCP may be evaluated by using monocyte-derived macrophages as effector cells and Daudi cells (ATCC® CCL-213™) or B cell leukemia or lymphoma tumor cells expressing CD38 as target cells engineered to express GFP or other labeled molecules. Effector:target cell ratio may be, for example, 4:1. Effector cells may be incubated with target cells for 4 hours with or without anti-CD38 antibody. After incubation, cells may be detached using accutase. Macrophages may be identified with anti-CD11b and anti-CD14 antibodies coupled to a fluorescent label, and percent phagocytosis may be determined based on % GFP fluorescence in the CD11⁺CD14⁺ macrophages using standard methods. Anti-CD38 antibodies used in the disclosed methods may induce ADCP by about 20%, 25%, 30%, 35%, 40%, 45%, 50%, 55%, 60%, 65%, 70%, 75%, 80%, 85%, 90%, 95% or 100%.

The Fc portion of the anti-CD38 antibody may mediate antibody effector functions such as antibody-dependent cell-mediated cytotoxicity (ADCC), antibody-dependent cellular phagocytosis (ADCP), or complement dependent cytotoxicity (CDC). Such functions may be mediated by binding of an Fc effector domain(s) to an Fc receptor on an immune cell with phagocytic or lytic activity, or by binding of an Fc effector domain(s) to components of the complement system. Typically, the effect(s) mediated by the Fc-binding cells or complement components result in inhibition and/or depletion of target cells, e.g., CD38-expressing cells. Human IgG isotypes IgG1, IgG2, IgG3, and IgG4 exhibit differential capacity for effector functions. ADCC may be mediated by IgG1 and IgG3, ADCP may be mediated by IgG1, IgG2, IgG3, and IgG4, and CDC may be mediated by IgG1 and IgG3.

ADCC elicited by the anti-CD38 antibodies may be enhanced by certain substitutions in the antibody Fc region. In some embodiments, the anti-CD38 antibodies comprise a substitution in the Fc region at amino acid position 256, 290, 298, 312, 356, 330, 333, 334, 360, 378, 430, or any combination thereof, wherein the residue numbering is according to the EU index (substitutions described in U.S. Pat. No. 6,737,056).

ADCC elicited by the anti-CD38 antibodies can also be enhanced by engineering an antibody oligosaccharide component. Human IgG1 or IgG3 are N-glycosylated at Asn297 with the majority of the glycans in the biantennary G0, G0F, G1, G1F, G2, or G2F forms. Antibodies produced by non-engineered CHO cells typically have a glycan fucose content (i.e. the amount of the fucose monosaccharide within the sugar chain at Asn297) of about at least 85%. The removal of the core fucose from the biantennary complex-type oligosaccharides attached to the Fc regions enhances the ADCC of antibodies via improved FcγRIIIa binding without altering antigen binding or CDC activity. Such modified antibodies can be achieved using different methods reported to lead to the successful expression of relatively high defucosylated antibodies bearing the biantennary complex-type of Fc oligosaccharides such as: control of culture osmolality (Konno et al., Cytotechnology 64:249-65, 2012); application of a variant CHO line Lec13 as the host cell line (Shields et al., J Biol Chem 277:26733-26740, 2002); application of a variant CHO line EB66 as the host cell line (Olivier et al., MAbs 2(4), 2010; Epub ahead of print; PMID:20562582); application of a rat hybridoma cell line YB2/0 as the host cell line (Shinkawa et al., J Biol Chem 278:3466-3473, 2003); introduction of small interfering RNA specifically against the α 1,6-fucosyltrasferase (FUT8) gene (Mori et al., Biotechnol Bioeng 88:901-908, 2004); or coexpression of β-1,4-N-acetylglucosaminyltransferase III and Golgi α-mannosidase II or a potent alpha-mannosidase I inhibitor, such as kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments, the anti-CD38 antibody can have a biantennary glycan structure with fucose content between about 0% to about 15%, for example 15%, 14%, 13%, 12%, 11%, 10%, 9%, 8%, 7%, 6%, 5%, 4%, 3%, 2%, 1%, or 0%. In some embodiments, the anti-CD38 antibody can have a biantennary glycan structure with fucose content of about 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0%. Substitutions in the Fc region and reduced fucose content may enhance the ADCC activity of the anti-CD38 antibody.

Fucose content may be characterized and quantified by multiple methods, for example: 1) using MALDI-TOF of N-glycosidase F treated sample (e.g. complex, hybrid, and oligo- and high-mannose structures) as described in Int'l Pat. Pub. No. WO2008/0775462); 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the antibody to constituent peptides by enzymatic digestion (e.g., trypsin or endopeptidase Lys-C), and subsequent separation, detection, and quantitation by HPLC-MS (UPLC-MS); and 5) separation of the antibody oligosaccharides from the antibody protein by specific enzymatic deglycosylation with PNGase F at Asn297. The oligosaccharides thus released can be labeled with a fluorophore, separated, and identified by various complementary techniques which allow: fine characterization of the glycan structures by matrix-assisted laser desorption ionization (MALDI) mass spectrometry by comparison of the experimental masses with the theoretical masses; determination of the degree of sialylation by ion exchange HPLC (GlycoSep C); separation and quantification of the oligosaccharide forms according to hydrophilicity criteria by normal-phase HPLC (GlycoSep N); and separation and quantification of the oligosaccharides by high performance capillary electrophoresis-laser induced fluorescence (HPCE-LIF).

The anti-CD38 antibody may bind human CD38 with a range of affinities (K_{D}). For example, the anti-CD38 antibody can bind CD38 with a K_{D} equal to or less than about 1×10⁻⁸ M, for example 5×10⁻⁹ M, 1×10⁻⁹ M, 5×10⁻¹⁰ M, 1×10⁻¹⁰ M, 5×10⁻¹¹ M, 1×10⁻¹¹ M, 5×10⁻¹² M, 1×10⁻¹² M, 5×10⁻¹³ M, 1×10⁻¹³ M, 5×10⁻¹⁴ M, 1×10⁻¹⁴ M, 5×10⁻¹⁵M, or any range or value therein, as determined by surface plasmon resonance or the Kinexa method, as practiced by those of skill in the art. In some embodiments, the anti-CD38 antibody can bind to CD38 with an affinity of equal to or less than 1×10⁻⁸ M. In some embodiments, the anti-CD38 antibody can bind to CD38 with an affinity of equal to or less than 1×10⁻⁹ M.

Antibody affinity can be measured using KinExA instrumentation, ELISA, or competitive binding assays known to those skilled in the art. The measured affinity of a particular antibody/CD38 interaction may vary if measured under different conditions (e.g., osmolarity, pH). Thus, measurements of affinity and other binding parameters (e.g., K_{D}, Kₒₙ, K_{off}) are typically made with standardized conditions and a standardized buffer. Those skilled in the art will appreciate that the internal error for affinity measurements for example using Biacore 3000 or ProteOn (measured as standard deviation, SD) may typically be within 5-33% for measurements within the typical limits of detection. Therefore, the term “about” in the context of K_{D} reflects the typical standard deviation in the assay. For example, the typical SD for a K_{D} of 1×10⁻⁹ M is up to ±0.33×10⁻⁹ M.

The dose of anti-CD38 antibody given to a subject having multiple myeloma is sufficient to alleviate or at least partially arrest the disease being treated (“therapeutically effective amount”) and includes from about 0.005 mg to about 100 mg/kg, e.g. about 0.05 mg to about 30 mg/kg or about 5 mg to about 25 mg/kg, or about 4 mg/kg, about 8 mg/kg, about 16 mg/kg, or about 24 mg/kg. Suitable doses include, for example, about 1, 2, 3, 4, 5, 6, 7, 8, 9, 10, 15, 16, 17, 18, 19, 20, 21, 22, 23, 24, 25, 30, 40, 50, 60, 70, 80, 90, or 100 mg/kg.

A fixed unit dose of the anti-CD38 antibody may also be given, for example, 50, 100, 200, 500, or 1000 mg, or the dose may be based on the patient's surface area, e.g., 500, 400, 300, 250, 200, or 100 mg/m². Usually between 1 and 8 doses, (e.g., 1, 2, 3, 4, 5, 6, 7, or 8) may be administered to treat MM, but 9, 10, 11, 12, 13, 14, 15, 16, 17, 18, 19, 20, or more doses may be given.

The administration of the anti-CD38 antibody may be repeated after one day, two days, three days, four days, five days, six days, one week, two weeks, three weeks, one month, five weeks, six weeks, seven weeks, two months, three months, four months, five months, six months, or longer. Repeated courses of treatment are also possible, as is chronic administration. The repeated administration may be at the same dose or at a different dose. For example, the anti-CD38 antibody may be administered at 8 mg/kg or at 16 mg/kg at weekly intervals for 8 weeks, followed by administration at 8 mg/kg or at 16 mg/kg every two weeks for an additional 16 weeks, followed by administration at 8 mg/kg or at 16 mg/kg every four weeks by intravenous infusion.

The anti-CD38 antibodies may be administered by maintenance therapy, such as, e.g., once a week for a period of 6 months or more. For example, the anti-CD38 antibodies may be provided as a daily dosage in an amount of about 0.1 mg/kg to about 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90, or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19, or 20 after initiation of treatment, or any combination thereof, using single or divided doses of every 24, 12, 8, 6, 4, or 2 hours, or any combination thereof.

The anti-CD38 antibodies may also be administered prophylactically in order to reduce the risk of developing multiple myeloma, delay the onset of the occurrence of an event in multiple myeloma progression, and/or reduce the risk of recurrence when multiple myeloma is in remission.

Exemplary corticosteroids include, for example, a glucocorticoid (cortisol, for example), prednisone, or dexamethasone. In some embodiments, the corticosteroid is dexamethasone. Thus, the methods can comprise administering to the subject a therapeutically effective amount of an anti-CD38 antibody, dexamethasone, and a non-corticosteroid chemotherapeutic agent for a time sufficient to treat the high-risk multiple myeloma.

In some embodiments, corticosteroid is administered about 80 mg weekly. In some embodiments, corticosteroid is administered about 40 mg weekly. In some embodiments, corticosteroid is administered twice a week. In some embodiments, corticosteroid is administered four times a week. In some embodiments, corticosteroid is administered once a week. In some embodiments, corticosteriod is administered orally. In some embodiments, corticosteroid is administered intravenously. In some embodiments, corticosteroid is dexamethasone.

Exemplary non-corticosteroid chemotherapeutic agents include glutamic acid derivatives or proteasome inhibitors. Exemplary glutamic acid derivatives include thalidomide (Thalomid®) or a thalidomide analog, e.g. CC-5013 (lenalidomide, Revlimid™), pomalidomide or CC4047 (Actimid™). In some embodiments, the glutamic acid derivative is lenalidomide. Thus, the methods can comprise administering to the subject a therapeutically effective amount of an anti-CD38 antibody, a corticosteroid, and lenalidomide for a time sufficient to treat the high-risk multiple myeloma.

In some embodiments, lenalinomide is administered between about 10 mg to about 25 mg once a day. In some embodiments, lenalinomide is administered about 25 mg once a day.

Exemplary proteasome inhibitors include bortezomib (Velcade®), carfilzomib, or ixazomib. In some embodiments, the proteasome inhibitor is bortezomib. Thus, the methods can comprise administering to the subject a therapeutically effective amount of an anti-CD38 antibody, a corticosteroid, and bortezomib for a time sufficient to treat the high-risk multiple myeloma.

In some embodiments, bortezomib is administered about 1.5 mg/m² once a week. In some embodiments, bortezomib is administered about 1.3 mg/m² once a week. In some embodiments, bortezomib is administered about 1.3 mg/m² to about 1.5 mg/m² once a week. In some embodiments, bortezomib is administered about 1.3 mg/m² twice a week. In some embodiments, bortezomib is administered by subcutaneous injection.

In some embodiments, the methods can comprise administering to the subject a therapeutically effective amount of an anti-CD38 antibody, dexamethasone, and lenalidomide for a time sufficient to treat the high-risk multiple myeloma. In some embodiments, the methods can comprise administering to the subject a therapeutically effective amount of an anti-CD38 antibody, dexamethasone, and bortezomib for a time sufficient to treat the high-risk multiple myeloma. In some embodiments, the methods can comprise administering to the subject a therapeutically effective amount of an anti-CD38 antibody, dexamethasone, lenalidomide, and bortezomib for a time sufficient to treat the high-risk multiple myeloma.

Subjects can be classified as “high-risk” if they have one or more of the following cytogenetic abnormalities: t(4;14)(p16;q32), t(14;16)(q32;q23), or del17p. Thus, the subject having high-risk multiple myeloma can have one or more chromosomal abnormalities comprising:

$$a . t ( 4 ; 14 ) ( p 16 ; q 32 ) ; \\ b . t ( 14 ; 16 ) ( q 32 ; q 23 ) ; \\ c . del 17 p ; \\ d . t ( 4 ; 14 ) ( p 16 ; q 32 ) and t ( 14 ; 16 ) ( q 32 ; q 23 ) ; \\ e . t ( 4 ; 14 ) ( p 16 ; q 32 ) and del 17 p ; \\ f . t ( 14 ; 16 ) ( q 32 ; q 23 ) and del 17 p ; or \\ g . t ( 4 ; 14 ) ( p 16 ; q 32 ) , t ( 14 ; 16 ) ( q 32 ; q 23 ) and del 17 p .$$

Subjects can be classified as “standard risk” if they have none of the following cytogenetic abnormalities: t(4;14)(p16;q32), t(14;16)(q32;q23), or del17p.

The cytogenetic abnormalities can be detected by fluorescent in situ hybridization (FISH). In both chromosomal translocations, an oncogene is translocated to the IgH region on chromosome 14q32, resulting in dysregulation of these genes. t(4;14)(p16;q32) involves translocation of fibroblast growth factor receptor 3 (FGFR3) and multiple myeloma SET domain containing protein (MMSET) (also called WHSC1/NSD2), and t(14;16)(q32;q23) involves translocation of the MAF transcription factor C-MAF. Deletion of 17p (del17p) involves loss of the p53 gene locus.

The subject can have naive multiple myeloma, relapsed multiple myeloma, or refractory multiple myeloma. In some embodiments, the subject has high risk refractory and/or relapsed multiple myeloma.

The methods of treatment can improve one or more outcome measurements of the subject compared to a subject receiving the corticosteroid and the non-corticosteroid chemotherapeutic agent. Exemplary outcome measurements comprise progression-free survival, overall response rate, very good partial response or better, complete response or better, or any combination thereof.

The methods can achieve minimal residual disease-negativity in the subject. The negative minimal residual disease status can be determined at a sensitivity of 0.01% (10⁻⁴), 0.001% (10⁻⁵), 0.0001% (10⁻⁶), or a combination thereof. The negative minimal residual disease can be detected by evaluating an amount of myeloma cells in a bone marrow aspirate sample from the subject.

The anti-CD38 antibody, corticosteroid, and non-corticosteroid chemotherapeutic agent may be administered over any convenient timeframe. In some embodiments, the anti-CD38 antibody, corticosteroid, and non-corticosteroid chemotherapeutic agent are administered simultaneously. In some embodiments, the anti-CD38 antibody, corticosteroid, and non-corticosteroid chemotherapeutic agent are administered sequentially in any order. An exemplary dosing schedule includes:
Daratumumab can be administered as an IV infusion at a dose of 16 mg/kg once per week (days 1, 8, and 15) for cycles 1-3, once every 4 weeks (on Day 1) during cycles 4-8, and once every 4 weeks thereafter. Bortezomib can be administered at a dose of 1.3 mg/m² subcutaneously (SC) on Days 1, 4, 8, and 11 of cycles 1-8. Dexamethasone can be administered orally at 20 mg on Days 1, 2, 4, 5, 8, 9, 11, and 12, for a total dose of 160 mg per cycle.
Daratumumab can be administered as an IV infusion at a dose of 16 mg/kg weekly (on days 1, 8, 15, and 22) for 8 weeks during cycles 1 and 2, every 2 weeks (on days 1 and 15) for 16 weeks (cycles 3 through 6), and every 4 weeks thereafter. Lenalidomide can be administered at a dose of 25 mg orally on days 1 to 21 of each cycle if the creatinine clearance is more than 60 ml per minute (or a dose of 10 mg daily if the creatinine clearance is 30 to 60 ml per minute) and dexamethasone at a dose of 40 mg weekly. For the daratumumab group, the dose of dexamethasone can be split: dexamethasone can be administered at a dose of 20 mg before infusion as prophylaxis for infusion-related reactions and 20 mg can be administered the next day.

The anti-CD38 antibody, corticosteroid, and non-corticosteroid chemotherapeutic agent may be administered together with any form of radiation therapy including external beam radiation, intensity modulated radiation therapy (IMRT) and any form of radiosurgery including Gamma Knife, Cyberknife, Linac, and interstitial radiation (e.g., implanted radioactive seeds, GliaSite balloon), and/or with surgery.

The anti-CD38 antibody, corticosteroid, and non-corticosteroid chemotherapeutic agent may be administered together with autologous hematopoietic stem cell transplant (AHSC).

### Methods of Achieving Negative Minimal Residual Disease Status in a Subject

Also provided are methods of achieving negative minimal residual disease status in a subject having multiple myeloma comprising administering to the subject a therapeutically effective amount of an anti-CD38 antibody, a corticosteroid, and a non-corticosteroid chemotherapeutic agent for a time sufficient to achieve negative minimal residual disease status.

The negative minimal residual disease status can be determined at a sensitivity of 0.01% (10⁻⁴), 0.001% (10⁻⁵), 0.0001% (10⁻⁶), or a combination thereof. In some embodiments, the negative minimal residual disease is detected by evaluating an amount of myeloma cells in a bone marrow aspirate sample from the subject.

In addition to achieving negative minimal residual disease status, the method also reduces progression-free survival events.

The subject can have naive multiple myeloma, relapsed multiple myeloma, or refractory multiple myeloma. In some embodiments, the subject has high-risk refractory and/or relapsed multiple myeloma. Subjects with high-risk multiple myeloma are known to relapse early and have poor prognosis and outcome.

In some embodiments, the subject has high-risk multiple myeloma. Subjects can be classified as “high risk” if they have one or more of the following cytogenetic abnormalities: t(4;14)(p16;q32), t(14;16)(q32;q23), or del17p. Thus, the subject having high-risk multiple myeloma can have one or more chromosomal abnormalities comprising:

$$a . t ( 4 ; 14 ) ( p 16 ; q 32 ) ; \\ b . t ( 14 ; 16 ) ( q 32 ; q 23 ) ; \\ c . del 17 p ; \\ d . t ( 4 ; 14 ) ( p 16 ; q 32 ) and t ( 14 ; 16 ) ( q 32 ; q 23 ) ; \\ e . t ( 4 ; 14 ) ( p 16 ; q 32 ) and del 17 p ; \\ f . t ( 14 ; 16 ) ( q 32 ; q 23 ) and del 17 p ; or \\ g . t ( 4 ; 14 ) ( p 16 ; q 32 ) , t ( 14 ; 16 ) ( q 32 ; q 23 ) and del 17 p .$$

Any of the anti-CD38 antibodies disclosed above for the methods of treatment can be used in the methods of achieving negative minimal residual disease status in a subject having multiple myeloma.

The anti-CD38 antibody can bind to a region of human CD38 comprising SKRNIQFSCKNIYR (SEQ ID NO: 2) and a region of human CD38 comprising EKVQTLEAWVIHGG (SEQ ID NO: 3).

The anti-CD38 antibody can comprise a heavy chain CDR1 comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 8, a light chain CDR1 comprising the amino acid sequence of SEQ ID NO: 9, a light chain CDR2 comprising the amino acid sequence of SEQ ID NO: 10, and a light chain CDR3 comprising the amino acid sequence of SEQ ID NO: 11. The anti-CD38 antibody can comprise a VH comprising an amino acid sequence that is 95%, 96%, 97%, 98%, 99%, or 100% identical to that of SEQ ID NO: 4 and a VL comprising an amino acid sequence that is 95%, 96%, 97%, 98%, 99%, or 100% identical to that of SEQ ID NO: 5. In some embodiments, the anti-CD38 antibody can comprise a VH comprising the amino acid sequence of SEQ ID NO: 4 and a VL comprising the amino acid sequence of SEQ ID NO: 5. The anti-CD38 antibody can comprise a heavy chain comprising the amino acid sequence of SEQ ID NO: 12 and a light chain comprising the amino acid sequence of SEQ ID NO: 13.

The anti-CD38 antibody can comprise a heavy chain CDR1, a heavy chain CDR2, and a heavy chain CDR3 of a VH comprising the amino acid sequence of SEQ ID NO: 14, and a light chain CDR1, a light chain CDR2, and a light chain CDR3 of a VL comprising the amino acid sequence of SEQ ID NOs:15. The anti-CD38 antibody can comprise a VH comprising an amino acid sequence that is 95%, 96%, 97%, 98%, 99%, or 100% identical to that of SEQ ID NO: 15 and a VL comprising an amino acid sequence that is 95%, 96%, 97%, 98%, 99%, or 100% identical to that of SEQ ID NO: 15. In some embodiments, the anti-CD38 antibody can comprise a VH comprising the amino acid sequence of SEQ ID NO: 14 and a VL comprising the amino acid sequence of SEQ ID NO:15. In some embodiments, for example, the anti-CD38 antibody can comprise mAb003 (described in U.S. Pat. No. 7,829,693, incorporated herein by reference).

The anti-CD38 antibody can comprise a heavy chain CDR1, a heavy chain CDR2, and a heavy chain CDR3 of a VH comprising the amino acid sequence of SEQ ID NO: 16, and a light chain CDR1, a light chain CDR2, and a light chain CDR3 of a VL comprising the amino acid sequence of SEQ ID NO: 17. The anti-CD38 antibody can comprise a VH comprising an amino acid sequence that is 95%, 96%, 97%, 98%, 99%, or 100% identical to that of SEQ ID NO: 16 and a VL comprising an amino acid sequence that is 95%, 96%, 97%, 98%, 99%, or 100% identical to that of SEQ ID NO: 17. In some embodiments, the anti-CD38 antibody can comprise a VH comprising the amino acid sequence of SEQ ID NO: 16 and a VL comprising the amino acid sequence of SEQ ID NO: 17. In some embodiments, for example, the anti-CD38 antibody can comprise mAb024 (described in U.S. Pat. No. 7,829,693, incorporated herein by reference).

The anti-CD38 antibody can comprise a heavy chain CDR1, a heavy chain CDR2, and a heavy chain CDR3 of a VH comprising the amino acid sequence of SEQ ID NO: 18, and a light chain CDR1, a light chain CDR2, and a light chain CDR3 of a VL comprising the amino acid sequence of SEQ ID NO: 19. The anti-CD38 antibody can comprise a VH comprising an amino acid sequence that is 95%, 96%, 97%, 98%, 99%, or 100% identical to that of SEQ ID NO: 18 and a VL comprising an amino acid sequence that is 95%, 96%, 97%, 98%, 99%, or 100% identical to that of SEQ ID NO: 19. In some embodiments, the anti-CD38 antibody can comprise a VH comprising the amino acid sequence of SEQ ID NO: 18 and a VL comprising the amino acid sequence of SEQ ID NO: 19. In some embodiments, for example, the anti-CD38 antibody can comprise MOR-202 (MOR-03087) (described in U.S. Pat. No. 8,088,896, incorporated herein by reference).

The anti-CD38 antibody can comprise a heavy chain CDR1, a heavy chain CDR2, and a heavy chain CDR3 of a VH comprising the amino acid sequence of SEQ ID NO: 20, and a light chain CDR1, a light chain CDR2, and a light chain CDR3 of a VL comprising the amino acid sequence of SEQ ID NO: 21. The anti-CD38 antibody can comprise a VH comprising an amino acid sequence that is 95%, 96%, 97%, 98%, 99%, or 100% identical to that of SEQ ID NO: 20 and a VL comprising an amino acid sequence that is 95%, 96%, 97%, 98%, 99%, or 100% identical to that of SEQ ID NO: 21. In some embodiments, the anti-CD38 antibody can comprise a VH comprising the amino acid sequence of SEQ ID NO: 20 and a VL comprising the amino acid sequence of SEQ ID NO: 21. In some embodiments, for example, the anti-CD38 antibody can comprise isatuximab (described in U.S. Pat. No. 8,153,765, incorporated herein by reference). In some aspects, the VH and the VL of isatuximab may be expressed as IgG1/κ.

Any of the corticosteroids and non-corticosteroid chemotherapeutic agents disclosed above for the methods of treatment can be used in the methods of achieving negative minimal residual disease status in a subject having multiple myeloma. Suitable corticosteroids include, for example, a glucocorticoid (cortisol, for example), prednisone, or dexamethasone. In some embodiments, the corticosteroid is dexamethasone. Suitable non-corticosteroid chemotherapeutic agents include glutamic acid derivatives or proteasome inhibitors. Exemplary glutamic acid derivatives include thalidomide (Thalomid®) or a thalidomide analog, e.g. CC-5013 (lenalidomide, Revlimid™), pomalidomide or CC4047 (Actimid™). In some embodiments, the glutamic acid derivative is lenalidomide. Suitable proteasome inhibitors include bortezomib (Velcade®), carfilzomib, or ixazomib. In some embodiments, the proteasome inhibitor is bortezomib.

### Methods of Predicting a Likelihood of or Decreasing a Risk of Relapse and or Disease Progression

Provided are methods of predicting a likelihood of relapse and/or disease progression in a subject having multiple myeloma and methods of decreasing a risk of relapse and/or disease progression in a subject having multiple myeloma.

The methods of predicting a likelihood of relapse and/or disease progression in a subject having multiple myeloma comprise:
measuring a minimal residual disease status in the subject, wherein the subject has received a therapeutically effective amount of an anti-CD38 antibody,
wherein a positive minimal residual disease status is indicative of a likelihood of relapse and/or disease progression.

The methods of predicting a likelihood of relapse and/or disease progression in a subject having multiple myeloma comprise:
measuring a minimal residual disease status in the subject, wherein the subject has received a therapeutically effective amount of an anti-CD38 antibody, a corticosteroid, and a non-corticosteroid chemotherapeutic agent,
wherein a positive minimal residual disease status is indicative of a likelihood of relapse and/or disease progression.

The methods of decreasing a risk of relapse and/or disease progression in a subject having multiple myeloma comprise:
administering to the subject a therapeutically effective amount of an anti-CD38 antibody, a corticosteroid, and a non-corticosteroid chemotherapeutic agent to achieve a negative minimal residual disease status, wherein the negative residual disease status is indicative of a decreased risk of relapse and/or disease progression.

The subject can have naive multiple myeloma, relapsed multiple myeloma, refractory multiple myeloma, or relapsed and refractory multiple myeloma. In some embodiments, the subject has high risk refractory, relapsed, or relapsed and refractory multiple myeloma.

In some embodiments, the subject has high risk multiple myeloma. Subjects can be classified as “high-risk” if they have one or more of the following cytogenetic abnormalities: t(4;14)(p16;q32), t(14;16)(q32;q23), or del17p. Thus, the subject having high-risk multiple myeloma can have one or more chromosomal abnormalities comprising:

$$a . t ( 4 ; 14 ) ( p 16 ; q 32 ) ; \\ b . t ( 14 ; 16 ) ( q 32 ; q 23 ) ; \\ c . del 17 p ; \\ d . t ( 4 ; 14 ) ( p 16 ; q 32 ) and t ( 14 ; 16 ) ( q 32 ; q 23 ) ; \\ e . t ( 4 ; 14 ) ( p 16 ; q 32 ) and del 17 p ; \\ f . t ( 14 ; 16 ) ( q 32 ; q 23 ) and del 17 p ; or \\ g . t ( 4 ; 14 ) ( p 16 ; q 32 ) , t ( 14 ; 16 ) ( q 32 ; q 23 ) and del 17 p .$$

Any of the anti-CD38 antibodies disclosed above for the methods of treatment can be used in the methods of predicting a likelihood of relapse and/or disease progression in a subject having multiple myeloma and methods of decreasing a risk of relapse and/or disease progression in a subject having multiple myeloma.

The anti-CD38 antibody can bind to a region of human CD38 comprising SKRNIQFSCKNIYR (SEQ ID NO: 2) and a region of human CD38 comprising EKVQTLEAWVIHGG (SEQ ID NO: 3).

The anti-CD38 antibody can comprise a heavy chain CDR1 comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 8, a light chain CDR1 comprising the amino acid sequence of SEQ ID NO: 9, a light chain CDR2 comprising the amino acid sequence of SEQ ID NO: 10, and a light chain CDR3 comprising the amino acid sequence of SEQ ID NO: 11. The anti-CD38 antibody can comprise a VH comprising an amino acid sequence that is 95%, 96%, 97%, 98%, 99%, or 100% identical to that of SEQ ID NO: 4 and a VL comprising an amino acid sequence that is 95%, 96%, 97%, 98%, 99%, or 100% identical to that of SEQ ID NO: 5. In some embodiments, the anti-CD38 antibody can comprise VH comprising the amino acid sequence of SEQ ID NO: 4 and VL comprising the amino acid sequence of SEQ ID NO: 5. The anti-CD38 antibody can comprise a heavy chain comprising the amino acid sequence of SEQ ID NO: 12 and a light chain comprising the amino acid sequence of SEQ ID NO: 13.

The anti-CD38 antibody can comprise a heavy chain CDR1, a heavy chain CDR2, and a heavy chain CDR3 of a VH comprising the amino acid sequence of SEQ ID NO: 14, and a light chain CDR1, a light chain CDR2, and a light chain CDR3 of a VL comprising the amino acid sequence of SEQ ID NO:15. The anti-CD38 antibody can comprise a VH comprising an amino acid sequence that is 95%, 96%, 97%, 98%, 99%, or 100% identical to that of SEQ ID NO: 15 and a VL comprising an amino acid sequence that is 95%, 96%, 97%, 98%, 99%, or 100% identical to that of SEQ ID NO: 15. In some embodiments, the anti-CD38 antibody can comprise a VH comprising the amino acid sequence of SEQ ID NO: 14 and a VL comprising the amino acid sequence of SEQ ID NOs:15. In some embodiments, for example, the anti-CD38 antibody can comprise mAb003 (described in U.S. Pat. No. 7,829,693, incorporated herein by reference).

The anti-CD38 antibody can comprise a heavy chain CDR1, a heavy chain CDR2, and a heavy chain CDR3 of a VH comprising the amino acid sequence of SEQ ID NO: 16, and a light chain CDR1, a light chain CDR2, and a light chain CDR3 of a VL comprising the amino acid sequence of SEQ ID NO: 17. The anti-CD38 antibody can comprise a VH comprising an amino acid sequence that is 95%, 96%, 97%, 98%, 99%, or 100% identical to that of SEQ ID NO: 16 and a VL comprising an amino acid sequence that is 95%, 96%, 97%, 98%, 99%, or 100% identical to that of SEQ ID NO: 17. In some embodiments, the anti-CD38 antibody can comprise a VH comprising the amino acid sequence of SEQ ID NO: 16 and a VL comprising the amino acid sequence of SEQ ID NO: 17. In some embodiments, for example, the anti-CD38 antibody can comprise mAb024 (described in U.S. Pat. No. 7,829,693, incorporated herein by reference).

The anti-CD38 antibody can comprise a heavy chain CDR1, a heavy chain CDR2, and a heavy chain CDR3 of a VH comprising the amino acid sequence of SEQ ID NO: 18, and a light chain CDR1, a light chain CDR2, and a light chain CDR3 of a VL comprising the amino acid sequence of SEQ ID NO: 19. The anti-CD38 antibody can comprise a VH comprising an amino acid sequence that is 95%, 96%, 97%, 98%, 99%, or 100% identical to that of SEQ ID NO: 18 and a VL comprising an amino acid sequence that is 95%, 96%, 97%, 98%, 99%, or 100% identical to that of SEQ ID NO: 19. In some embodiments, the anti-CD38 antibody can comprise a VH comprising the amino acid sequence of SEQ ID NO: 18 and a VL comprising the amino acid sequence of SEQ ID NO: 19. In some embodiments, for example, the anti-CD38 antibody can comprise MOR-202 (MOR-03087) (described in U.S. Pat. No. 8,088,896, incorporated herein by reference).

The anti-CD38 antibody can comprise a heavy chain CDR1, a heavy chain CDR2, and a heavy chain CDR3 of a VH comprising the amino acid sequence of SEQ ID NO: 20, and a light chain CDR1, a light chain CDR2, and a light chain CDR3 of a VL comprising the amino acid sequence of SEQ ID NO: 21. The anti-CD38 antibody can comprise a VH comprising an amino acid sequence that is 95%, 96%, 97%, 98%, 99%, or 100% identical to that of SEQ ID NO: 20 and a VL comprising an amino acid sequence that is 95%, 96%, 97%, 98%, 99%, or 100% identical to that of SEQ ID NO: 21. In some embodiments, the anti-CD38 antibody can comprise a VH comprising the amino acid sequence of SEQ ID NO: 20 and a VL comprising the amino acid sequence of SEQ ID NO: 21. In some embodiments, for example, the anti-CD38 antibody can comprise isatuximab (described in U.S. Pat. No. 8,153,765, incorporated herein by reference). In some aspects, the VH and the VL of isatuximab may be expressed as IgG1/κ.

Any of the corticosteroids and non-corticosteroid chemotherapeutic agents disclosed above for the methods of treatment can be used in the methods of predicting a likelihood of relapse and/or disease progression in a subject having multiple myeloma and methods of decreasing a risk of relapse and/or disease progression in a subject having multiple myeloma. Suitable corticosteroids include, for example, a glucocorticoid (cortisol, for example), prednisone, or dexamethasone. In some embodiments, the corticosteroid is dexamethasone. Suitable non-corticosteroid chemotherapeutic agents include glutamic acid derivatives or proteasome inhibitors. Exemplary glutamic acid derivatives include thalidomide (Thalomid®) or a thalidomide analog, e.g. CC-5013 (lenalidomide, Revlimid™), pomalidomide or CC4047 (Actimid™). In some embodiments, the glutamic acid derivative is lenalidomide. Suitable proteasome inhibitors include bortezomib (Velcade®), carfilzomib, or ixazomib. In some embodiments, the proteasome inhibitor is bortezomib.

A description of example embodiments follows.

Embodiment 1. A method of achieving negative minimal residual disease status in a subject having multiple myeloma comprising administering to the subject a therapeutically effective amount of an anti-CD38 antibody, a corticosteroid, and a non-corticosteroid chemotherapeutic agent for a time sufficient to achieve negative minimal residual disease status.

Embodiment 2. The method of Embodiment 1, wherein the anti-CD38 antibody comprises a heavy chain CDR1 comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 8, a light chain CDR1 comprising the amino acid sequence of SEQ ID NO: 9, a light chain CDR2 comprising the amino acid sequence of SEQ ID NO: 10, and a light chain CDR3 comprising the amino acid sequence of SEQ ID NO: 11.

Embodiment 3. The method of Embodiments 1-2, wherein the corticosteroid is dexamethasone.

Embodiment 4. The method of Embodiments 1-3, wherein the non-corticosteroid chemotherapeutic agent is a glutamic acid derivative or a proteasome inhibitor.

Embodiment 5. The method of Embodiment 4, wherein the glutamic acid derivative is lenalidomide.

Embodiment 6. The method of Embodiment 4, wherein the proteasome inhibitor is bortezomib.

Embodiment 7. The method of Embodiment 5, wherein
the anti-CD38 antibody is administered as an intravenous infusion at a dose of about 16 mg/kg once per week in a 28-day cycle on days 1, 8, 15, and 22 for cycles 1 and 2, once every 2 weeks in a 28-day cycle on days 1 and 15 during cycles 3 through 6, and once every 4 weeks thereafter;
lenalidomide is administered at a dose of between about 10 mg to about 25 mg orally in a 28-day cycle on days 1 to 21; and
dexamethasone is administered at a dose of between about 20 mg to about 40 mg weekly.

Embodiment 8. The method of Embodiment 6, wherein
the anti-CD38 antibody is administered as an intravenous infusion at a dose of about 16 mg/kg once per week in a 21-day cycle on days 1, 8, and 15 for cycles 1-3, once every 3 weeks in a 21-day cycle on day 1 for cycles 4-8, and once every 4 weeks thereafter; and
bortezomib is administered at a dose of about 1.3 mg/m2 subcutaneously (SC) in a 21-day cycle on days 1, 4, 8, and 11 for cycles 1-8; and dexamethasone is administered at a dose of between about 20 mg to about 40 mg weekly.

Embodiment 9. The method of Embodiment 7, wherein dexamethasone is administered at 20 mg on days 1, 2, 4, 5, 8, 9, 11, and 12 for a total dose of 160 mg per cycle IV or PO.

Embodiment 10. The method of Embodiment 8, wherein dexamethasone is administered at 20 mg on days 1, 2, 4, 5, 8, 9, 11, and 12 for a total dose of 160 mg per cycle IV or PO.

Embodiment 11. The method of Embodiments 1-10, wherein the subject has relapsed or refractory multiple myeloma.

Embodiment 12. The method of Embodiments 1-11, wherein the subject has high risk multiple myeloma.

Embodiment 13. The method of Embodiment 12, wherein the subject having high-risk multiple myeloma has one or more chromosomal abnormalities comprising:

$$t ( 4 ; 14 ) ( p 16 ; q 32 ) ; \\ t ( 14 ; 16 ) ( q 32 ; q 23 ) ; \\ del 17 p ; \\ t ( 4 ; 14 ) ( p 16 ; q 32 ) and t ( 14 ; 16 ) ( q 32 ; q 23 ) ; \\ t ( 4 ; 14 ) ( p 16 ; q 32 ) and del 17 p ; \\ t ( 14 ; 16 ) ( q 32 ; q 23 ) and del 17 p ; or \\ t ( 4 ; 14 ) ( p 16 ; q 32 ) , t ( 14 ; 16 ) ( q 32 ; q 23 ) and del 17 p .$$

Embodiment 14. The method of Embodiments 1-13, wherein the negative minimal residual disease status is determined at a sensitivity of 0.01%, 0.001%, 0.0001%, or a combination thereof.

Embodiment 15. The method of Embodiments 1-14, wherein the anti-CD38 antibody binds to a region of human CD38 comprising SEQ ID NO: 2 and a region of human CD38 comprising SEQ ID NO: 3.

Embodiment 16. The method of Embodiments 1-15, wherein the anti-CD38 antibody comprises a heavy chain variable region comprising the amino acid sequence of SEQ ID NO: 4 and a light chain variable region comprising the amino acid sequence of SEQ ID NO: 5.

Embodiment 17. The method of Embodiments 1-16, wherein the anti-CD38 antibody comprises a heavy chain comprising the amino acid sequence of SEQ ID NO: 12 and a light chain comprising the amino acid sequence of SEQ ID NO: 13.

Embodiment 18. The method of Embodiments 1-13, wherein the anti-CD38 antibody comprises a heavy chain CDR1, a heavy chain CDR2, a heavy chain CDR3, a light chain CDR1, a light chain CDR2 and a light chain CDR3 of:
a VH comprising the amino acid sequence of SEQ ID NO: 14 and a VL comprising the amino acid sequence of SEQ ID NO: 15;
a VH comprising the amino acid sequence of SEQ ID NO: 16 and a VL comprising the amino acid sequence of SEQ ID NO: 17;
a VH comprising the amino acid sequence of SEQ ID NO: 18 and a VL comprising the amino acid sequence of SEQ ID NO: 19; or
a VH comprising the amino acid sequence of SEQ ID NO: 20 and a VL comprising the amino acid sequence of SEQ ID NO: 21.

Embodiment 19. The method of Embodiment 18, wherein the anti-CD38 antibody comprises:
a VH comprising the amino acid sequence of SEQ ID NO: 14 and a VL comprising the amino acid sequence of SEQ ID NO: 15;
a VH comprising the amino acid sequence of SEQ ID NO: 16 and a VL comprising the amino acid sequence of SEQ ID NO: 17;
a VH comprising the amino acid sequence of SEQ ID NO: 18 and a VL comprising the amino acid sequence of SEQ ID NO: 19; or
a VH comprising the amino acid sequence of SEQ ID NO: 20 and a VL comprising the amino acid sequence of SEQ ID NO: 21.

Embodiment 20. The method of Embodiments 1-2, wherein the corticosteroid is dexamethasone or prednisone.

Embodiment 21. The method of Embodiments 1-20, wherein the negative minimal residual disease is detected by evaluating an amount of myeloma cells in a bone marrow aspirate sample from the subject.

Embodiment 22. The method of Embodiments 1-21, wherein the method also reduces progression-free survival events.

Embodiment 23. The method of Embodiment 4, wherein the glutamic acid derivative is lenalidomide, thalidomide, or pomalidomide.

Embodiment 24. The method of Embodiment 4, wherein the proteasome inhibitor is bortezomib, carfilzomib, or ixazomib.

Embodiment 25. A method of treating a subject having high-risk multiple myeloma, comprising administering to the subject a therapeutically effective amount of an anti-CD38 antibody, a corticosteroid, and a non-corticosteroid chemotherapeutic agent for a time sufficient to treat the high-risk multiple myeloma.

Embodiment 26. The method of Embodiment 25, wherein the anti-CD38 antibody comprises a heavy chain CDR1 comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 8, a light chain CDR1 comprising the amino acid sequence of SEQ ID NO: 9, a light chain CDR2 comprising the amino acid sequence of SEQ ID NO: 10, and a light chain CDR3 comprising the amino acid sequence of SEQ ID NO: 11.

Embodiment 27. The method of Embodiments 25-26, wherein the corticosteroid is dexamethasone.

Embodiment 28. The method of Embodiments 25-27, wherein the non-corticosteroid chemotherapeutic agent is a glutamic acid derivative or a proteasome inhibitor.

Embodiment 29. The method of Embodiment 28, wherein the glutamic acid derivative is lenalidomide.

Embodiment 30. The method of Embodiment 28, wherein the proteasome inhibitor is bortezomib.

Embodiment 31. The method of Embodiment 29, wherein
the anti-CD38 antibody is administered as an intravenous infusion at a dose of about 16 mg/kg once per week in a 28-day cycle on days 1, 8, 15, and 22 for cycles 1 and 2, once every 2 weeks in a 28-day cycle on days 1 and 15 during cycles 3 through 6, and once every 4 weeks thereafter; and
lenalidomide is administered at a dose of between about 10 mg to about 25 mg orally in a 28-day cycle on days 1 to 21; and
dexamethasone is administered at a dose of between about 20 mg to about 40 mg weekly.

Embodiment 32. The method of Embodiment 30, wherein
the anti-CD38 antibody is administered as an intravenous infusion at a dose of about 16 mg/kg once per week in a 21-day cycle on days 1, 8, and 15 for cycles 1-3, once every 3 weeks in a 21-day cycle on day 1 for cycles 4-8, and once every 4 weeks thereafter; and
bortezomib is administered at a dose of about 1.3 mg/m2 subcutaneously (SC) in a 21-day cycle on days 1, 4, 8, and 11 for cycles 1-8; and
dexamethasone is administered at a dose of between about 20 mg to about 40 mg weekly.

Embodiment 33. The method of Embodiment 31, wherein dexamethasone is administered at 20 mg on days 1, 2, 4, 5, 8, 9, 11, and 12 for a total dose of 160 mg per cycle IV or PO.

Embodiment 34. The method of Embodiment 32, wherein dexamethasone is administered at 20 mg on days 1, 2, 4, 5, 8, 9, 11, and 12 for a total dose of 160 mg per cycle IV or PO.

Embodiment 35. The method of Embodiments 25-34, wherein the anti-CD38 antibody binds to a region of human CD38 comprising SEQ ID NO: 2 and a region of human CD38 comprising SEQ ID NO: 3.

Embodiment 36. The method of Embodiments 25-35, wherein the anti-CD38 antibody comprises a heavy chain variable region comprising the amino acid sequence of SEQ ID NO: 4 and a light chain variable region comprising the amino acid sequence of SEQ ID NO: 5.

Embodiment 37. The method of Embodiments 25-36, wherein the anti-CD38 antibody comprises a heavy chain comprising the amino acid sequence of SEQ ID NO: 12 and a light chain comprising the amino acid sequence of SEQ ID NO: 13.

Embodiment 38. The method of Embodiment 25, wherein the anti-CD38 antibody comprises a heavy chain CDR1, a heavy chain CDR2, a heavy chain CDR3, a light chain CDR1, a light chain CDR2 and a light chain CDR3 of: a VH comprising the amino acid sequence of SEQ ID NO: 14 and a VL comprising the amino acid sequence of SEQ ID NO: 15; a VH comprising the amino acid sequence of SEQ ID NO: 16 and a VL comprising the amino acid sequence of SEQ ID NO: 17; a VH comprising the amino acid sequence of SEQ ID NO: 18 and a VL comprising the amino acid sequence of SEQ ID NO: 19; or a VH comprising the amino acid sequence of SEQ ID NO: 20 and a VL comprising the amino acid sequence of SEQ ID NO: 21.

Embodiment 39. The method of Embodiment 38, wherein the anti-CD38 antibody comprises:
a VH comprising the amino acid sequence of SEQ ID NO: 14 and a VL comprising the amino acid sequence of SEQ ID NO: 15;
a VH comprising the amino acid sequence of SEQ ID NO: 16 and a VL comprising the amino acid sequence of SEQ ID NO: 17;
a VH comprising the amino acid sequence of SEQ ID NO: 18 and a VL comprising the amino acid sequence of SEQ ID NO: 19; or
a VH comprising the amino acid sequence of SEQ ID NO: 20 and a VL comprising the amino acid sequence of SEQ ID NO: 21.

Embodiment 40. The method of Embodiment 25, wherein the corticosteroid is dexamethasone or prednisone.

Embodiment 41. The method of Embodiment 25, wherein the corticosteroid is dexamethasone.

Embodiment 42. The method of Embodiment 25, wherein the non-corticosteroid chemotherapeutic agent is a glutamic acid derivative or a proteasome inhibitor.

Embodiment 43. The method of Embodiment 42, wherein the glutamic acid derivative is lenalidomide, thalidomide, or pomalidomide.

Embodiment 44. The method of Embodiment 42, wherein the glutamic acid derivative is lenalidomide.

Embodiment 45. The method of Embodiment 42, wherein the proteasome inhibitor is bortezomib, carfilzomib, or ixazomib.

Embodiment 46. The method of Embodiment 42, wherein the proteasome inhibitor is bortezomib.

Embodiment 47. The method of Embodiment 25, wherein the subject having high-risk multiple myeloma has one or more chromosomal abnormalities comprising:

$$t ( 4 ; 14 ) ( p 16 ; q 32 ) ; \\ t ( 14 ; 16 ) ( q 32 ; q 23 ) ; \\ del 17 p ; \\ t ( 4 ; 14 ) ( p 16 ; q 32 ) and t ( 14 ; 16 ) ( q 32 ; q 23 ) ; \\ t ( 4 ; 14 ) ( p 16 ; q 32 ) and del 17 p ; \\ t ( 14 ; 16 ) ( q 32 ; q 23 ) and del 17 p ; or \\ t ( 4 ; 14 ) ( p 16 ; q 32 ) , t ( 14 ; 16 ) ( q 32 ; q 23 ) and del 17 p .$$

Embodiment 48. The method of Embodiment 25, wherein the subject has high risk refractory or relapsed multiple myeloma.

Embodiment 49. The method of Embodiment 25, wherein the method improves one or more outcome measurements of the subject compared to a subject receiving the corticosteroid and the non-corticosteroid chemotherapeutic agent.

Embodiment 50. The method of Embodiment 49, wherein the one or more outcome measurements comprise progression-free survival, overall response rate, very good partial response or better, complete response or better, or any combination thereof.

Embodiment 51. The method of Embodiment 25, wherein the method achieves minimal residual disease-negativity in the subject.

Embodiment 52. A method of decreasing a risk of relapse and/or disease progression in a subject having multiple myeloma comprising:
administering to the subject a therapeutically effective amount of an anti-CD38 antibody, a corticosteroid, and a non-corticosteroid chemotherapeutic agent to achieve a negative minimal residual disease status, wherein the negative residual disease status is indicative of a decreased risk of relapse and/or disease progression.

Embodiment 53. A method of predicting a likelihood of relapse and/or disease progression in a subject having multiple myeloma comprising:
measuring a minimal residual disease status in the subject, wherein the subject has received a therapeutically effective amount of an anti-CD38 antibody, a corticosteroid, and a non-corticosteroid chemotherapeutic agent,
wherein a positive minimal residual disease status is indicative of a likelihood of relapse and/or disease progression.

Embodiment 54. The method of Embodiments 52-53, wherein the anti-CD38 antibody comprises a heavy chain CDR1 comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 8, a light chain CDR1 comprising the amino acid sequence of SEQ ID NO: 9, a light chain CDR2 comprising the amino acid sequence of SEQ ID NO: 10, and a light chain CDR3 comprising the amino acid sequence of SEQ ID NO: 11.

Embodiment 55. The method of Embodiments 52-54, wherein the corticosteroid is dexamethasone.

Embodiment 56. The method of Embodiments 52-55, wherein the non-corticosteroid chemotherapeutic agent is a glutamic acid derivative or a proteasome inhibitor.

Embodiment 57. The method of Embodiment 56, wherein the glutamic acid derivative is lenalidomide.

Embodiment 58. The method of Embodiment 56, wherein the proteasome inhibitor is bortezomib.

Embodiment 59. The method of Embodiment 57, wherein
the anti-CD38 antibody is administered as an intravenous infusion at a dose of about 16 mg/kg once per week in a 28-day cycle on days 1, 8, 15, and 22 for cycles 1 and 2, once every 2 weeks in a 28-day cycle on days 1 and 15 during cycles 3 through 6, and once every 4 weeks thereafter;
lenalidomide is administered at a dose of between about 10 mg to about 25 mg orally in a 28-day cycle on days 1 to 21; and
dexamethasone is administered at a dose of between about 20 mg to about 40 mg weekly.

Embodiment 60. The method of Embodiment 58, wherein
the anti-CD38 antibody is administered as an intravenous infusion at a dose of about 16 mg/kg once per week in a 21-day cycle on days 1, 8, and 15 for cycles 1-3, once every 3 weeks in a 21-day cycle on day 1 for cycles 4-8, and once every 4 weeks thereafter; and
bortezomib is administered at a dose of about 1.3 mg/m2 subcutaneously (SC) in a 21-day cycle on days 1, 4, 8, and 11 for cycles 1-8; and
dexamethasone is administered at a dose of between about 20 mg to about 40 mg weekly.

Embodiment 61. The method of Embodiments 55-60, wherein dexamethasone is administered at 20 mg on days 1, 2, 4, 5, 8, 9, 11, and 12 for a total dose of 160 mg per cycle IV or PO.

Embodiment 62. The method of Embodiments 52-61, wherein the subject has relapsed or refractory multiple myeloma.

Embodiment 63. The method of Embodiments 52-62, wherein the subject has high risk multiple myeloma.

Embodiment 64. The method of Embodiment 63, wherein the subject having high-risk multiple myeloma has one or more chromosomal abnormalities comprising:

$$t ( 4 ; 14 ) ( p 16 ; q 32 ) ; \\ t ( 14 ; 16 ) ( q 32 ; q 23 ) ; \\ del 17 p ; \\ t ( 4 ; 14 ) ( p 16 ; q 32 ) and t ( 14 ; 16 ) ( q 32 ; q 23 ) ; \\ t ( 4 ; 14 ) ( p 16 ; q 32 ) and del 17 p ; \\ t ( 14 ; 16 ) ( q 32 ; q 23 ) and del 17 p ; or \\ t ( 4 ; 14 ) ( p 16 ; q 32 ) , t ( 14 ; 16 ) ( q 32 ; q 23 ) and del 17 p .$$

Embodiment 65. The method of Embodiments 52-64, wherein the anti-CD38 antibody binds to a region of human CD38 comprising SEQ ID NO: 2 and a region of human CD38 comprising SEQ ID NO: 3.

Embodiment 66. The method of Embodiments 52-65, wherein the anti-CD38 antibody comprises a heavy chain variable region comprising the amino acid sequence of SEQ ID NO: 4 and a light chain variable region comprising the amino acid sequence of SEQ ID NO: 5.

Embodiment 67. The method of Embodiments 52-66, wherein the anti-CD38 antibody comprises a heavy chain comprising the amino acid sequence of SEQ ID NO: 12 and a light chain comprising the amino acid sequence of SEQ ID NO: 13.

Embodiment 68. The method of Embodiments 52-64, wherein the anti-CD38 antibody comprises a heavy chain CDR1, a heavy chain CDR2, a heavy chain CDR3, a light chain CDR1, a light chain CDR2 and a light chain CDR3 of:
a VH comprising the amino acid sequence of SEQ ID NO: 14 and a VL comprising the amino acid sequence of SEQ ID NO: 15;
a VH comprising the amino acid sequence of SEQ ID NO: 16 and a VL comprising the amino acid sequence of SEQ ID NO: 17;
a VH comprising the amino acid sequence of SEQ ID NO: 18 and a VL comprising the amino acid sequence of SEQ ID NO: 19; or
a VH comprising the amino acid sequence of SEQ ID NO: 20 and a VL comprising the amino acid sequence of SEQ ID NO: 21.

Embodiment 69. The method of Embodiment 68, wherein the anti-CD38 antibody comprises:
a VH comprising the amino acid sequence of SEQ ID NO: 14 and a VL comprising the amino acid sequence of SEQ ID NO: 15;
a VH comprising the amino acid sequence of SEQ ID NO: 16 and a VL comprising the amino acid sequence of SEQ ID NO: 17;
a VH comprising the amino acid sequence of SEQ ID NO: 18 and a VL comprising the amino acid sequence of SEQ ID NO: 19; or
a VH comprising the amino acid sequence of SEQ ID NO: 20 and a VL comprising the amino acid sequence of SEQ ID NO: 21.

Embodiment 70. The method of Embodiments 52-54, wherein the corticosteroid is dexamethasone or prednisone.

Embodiment 71. The method of Embodiment 56, wherein the glutamic acid derivative is lenalidomide, thalidomide, or pomalidomide.

Embodiment 72. The method of Embodiment 56, wherein the proteasome inhibitor is bortezomib, carfilzomib, or ixazomib.

### EXAMPLES

The following examples are provided to further describe some of the embodiments disclosed herein. The examples are intended to illustrate, not to limit, the disclosed embodiments.

### Example 1. Study Design NCT02136134 (Castor)

The purpose of this study is to assess the effects of administration of daratumumab when combined with VELCADE® (bortezomib) and dexamethasone compared with VELCADE® (bortezomib) and dexamethasone alone, for participants with relapsed or refractory multiple myeloma. The study was a multicenter, randomized open-label active-controlled phase 3 trial. A prespecified interim analysis has been described in Palumpo et al., NEJM 375:754-66, 2016. The clinical trial number for this study is NCT02136134.

### Eligibility

Patients with documented progressive multiple myeloma (according to International Myeloma Working Group IMWG criteria) who had received at least one previous therapy for multiple myeloma and had at least a partial response to the at least one previous therapy were eligible.

### Exclusion Criteria

Patients who had received daratumumab or other anti-CD38 therapies previously, patients who were refractory to or who had unacceptable side effects from bortezomib, patients with a neutrophil count<1000 cells/mm³, hemoglobin<7.5 g/dl, platelet count<75,000/mm³, creatinine clearance<20 ml/min per 1.73 m² body-surface area, alanine aminotransferase or aspartate aminotransferase level of 2.5 or more times the upper limit of the normal range, and bilirubin level or 1.5 or more times the upper limit of the normal range, patients who had disease that was refractory to another proteasome inhibitor, or patients who had grade 2 or higher peripheral neutropathy or neuropathic pain were excluded from the study.

### Trial Treatments

498 patients were randomly assigned in a 1:1 ratio to receive daratumumab, bortezomib and dexamethasone (DVd; “daratumumab group”) or bortezomib and dexamethasone (Vd; “control group”). Randomization was stratified by International Staging System (ISS), number of prior treatment programs (1 vs. 2 or 3 vs. >3), and prior VELCADE® treatment (“no” vs. “yes”).

Daratumumab was administered as an IV infusion at a dose of 16 mg/kg once per week (days 1, 8, and 15) for cycles 1-3, once every 3 weeks (on Day 1) during cycles 4-8, and once every 4 weeks thereafter. VELCADE® was administered at a dose of 1.3 mg/m² subcutaneously (SC) on Days 1, 4, 8, and 11 of cycles 1-8. Dexamethasone was administered orally at 20 mg on Days 1, 2, 4, 5, 8, 9, 11, and 12, for a total dose of 160 mg per cycle.

### Primary Outcome Measures

Progression-free survival (PFS), defined as the time from the date of randomization to the date of disease progression or death, whichever occurred first.

### Secondary Outcome Measures

Time to disease progression (TTP), the overall response rate (ORR), the proportion of patients with very good partial response (VGPR) or better, the duration of response, the time to response and overall survival (OS). TTP is defined as the time from the date of randomization to the date of first documented evidence of progression, as defined in the International Myeloma Working Group (IMWG) criteria. The Overall Response is defined a stringent complete response (sCR), complete response, very good partial response (VGPR), or partial response (PR) as per IMWG Criteria. Duration of response will be calculated from the date of initial documentation of a response to the date of first documented evidence of progressive disease, as defined in the IMWG criteria. Time to response is defined as the time from the date of first dose of study treatment to the date of the first documentation of observed response. VGPR is defined as a greater than 90% reduction in blood myeloma protein (M-protein) plus urine myeloma protein less than 100 mg per 24 hours. OS will be measured from the date of randomization to the date of the participant's death.

In addition, percentage of participants with Minimal Residual Disease (MRD) will be assessed, in participants who achieve ≥VGPR by analyzing bone marrow aspiration specimens.

### Safety Assessments

Safety assessments included the evaluation of adverse events, clinical laboratory testing, vital signs, and electrocardiography. The adverse events were graded according to the National Cancer Institute Common Terminology Criteria for Adverse Events, version 4.03.

### Statistics

A group sequential design with one prespecified interim analysis was used to evaluate the primary end point. The O'Brien-Fleming stopping boundary at the time of the interim analysis for the primary end point was calculated with the use of a Lan-DeMets alpha-spending function on the basis of the number of events observed at the data-cutoff date. Efficacy analyses were based on the intention-to-treat population, including all patients who underwent randomization. The secondary end points were compared between the DVd and the Vd with the use of a stratified lorank test. Hazard ratios and corresponding 95% confidence intervals were estimated with the use of a stratified Cox regression model, with treatment as the sole explanatory variable. The Kaplan-Meier method was used to estimate the distribution. A stratified Cochran-Mantel-Haenszel chi-square test was used to test between-group differences.

### Interim Results at Data-Cutoff Date of Jan. 11, 2016

At the time of the data-cutoff date, 243 patients in the DVd and 237 patients in the Vd had received at least one dose of trial treatment. 74 patients in the DVd and 104 in the Vd had discontinued treatment because of progressive disease or adverse events. Patient demographic, disease and clinical characteristics in the intention-to-treat patients in the two groups are shown in Table 1. International Staging System (ISS) disease staging was derived on the basis of the combination of serum β2-microglobulin and albumin. The ISS consists of three stages: stage I, serum β2-microglobulin level lower than 3.5 mg per liter (300 nmol per liter) and albumin level of 3.5 g per deciliter or higher; stage II, neither stage I nor III; and stage III, serum β2-microglobulin of 5.5 mg per liter or higher (470 nmol per liter). Higher stages indicate more severe disease.

### Efficacy

The overall response rate was 82.9% in the DVd and 63.2% in the Vd (p<0.001). Table 2 shows the summary of responses among patients who could be evaluated for response.

TABLE 1

| | DVd | | Vd | |
|---|---|---|---|---|
| Characteristic | (N = 251) | | (N = 247) | |
| Age | | | | |
| Median (range)-yr | 64 | (30-88) | 64 | (33-85) |
| Distribution-no. (%) | | | | |
| <65 yr | 132 | (52.6) | 125 | (50.6) |
| 65-74 yr | 96 | (38.2) | 87 | (35.2) |
| ≥75 yr | 23 | (9.2) | 35 | (14.2) |
| Type of measurable | | | | |
| disease-no. (%) | | | | |
| IgG | 125 | (49.8) | 138 | (55.9) |
| IgA | 56 | (22.3) | 54 | (21.9) |
| Other | 5 | (2.0) | 4 | (1.6) |
| Detected in urine only | 40 | (15.9) | 36 | (14.6) |
| Detected in serum free | 25 | (10.0) | 14 | (5.7) |
| light-chains only | | | | |
| Not evaluated | 0 | | 1 | (0.4) |
| ISS disease staging-no. (%) | | | | |
| I | 98 | (39.0) | 96 | (38.9) |
| II | 94 | (37.5) | 100 | (40.5) |
| III | 59 | (23.5) | 51 | (20.6) |
| Cytogenetic profile-no. (%) | | | | |
| Standard-risk | 140/181 | (77.3) | 137/174 | (78.7) |
| cytogenetic abnormality | | | | |
| High-risk cytogenetic | 41/181 | (22.7) | 37/174 | (21.3) |
| abnormality | | | | |
| Del17p | 28/181 | (15.5) | 21/174 | (12.1) |
| t(4; 14) | 14/181 | (7.7) | 15/174 | (8.6) |
| t(14; 16) | 4/181 | (2.2) | 5/174 | (2.9) |
| Median time since | 3.87 | (0.7-20.7) | 3.72 | (0.6-18.6) |
| initial diagnosis | | | | |
| of multiple myeloma | | | | |
| (range)-yr | | | | |
| Number of previous lines | | | | |
| of therapy-no. (%) | | | | |
| 1 | 122 | (48.6) | 113 | (45.7) |
| 2 | 70 | (27.9) | 74 | (30.0) |
| 3 | 37 | (14.7) | 32 | (13.0) |
| >3 | 22 | (8.8) | 28 | (11.3) |
| Median no. of previous lines | 2 | (1-9) | 2 | (1-10) |
| of therapy (range) | | | | |
| Previous autologous stem-cell | 156 | (62.2) | 149 | (60.3) |
| transplantation-no. (%) | | | | |
| Previous alkylating agent | 240 | (95.6) | 224 | (90.7) |
| therapy-no. (%) | | | | |
| Previous proteasome inhibitor | 169 | (67.3) | 172 | (69.6) |
| therapy-no. (%) | | | | |
| Previous immunomodulatory | 179 | (71.3) | 198 | (80.2) |
| drug therapy-no. (%) | | | | |
| Previous proteasome | 112 | (44.6) | 129 | (52.2) |
| inhibitor + immuno- | | | | |
| modulatory drug | | | | |
| therapy-no. (%) | | | | |
| Disease refractory to last line | 76 | (30.3) | 85 | (34.4) |
| of therapy-no. (%) | | | | |

TABLE 2

| Response Category | DVd (N = 240) | | Vd (N = 234) | | P Value |
|---|---|---|---|---|---|
| Overall response | | | | | |
| No. with response | 199 | | 148 | | |
| Rate-% (95% CI) | 82.9 | (77.5-87.5) | 63.2 | (56.7-69.4) | <0.001 |
| Best overall response-no. (%) | | | | | |
| Complete response or better | 46 | (19.2) | 21 | (9.0) | 0.001 |
| Complete response | 35 | (14.6) | 16 | (6.8) | |
| Stringent complete response | 11 | (4.6) | 5 | (2.1) | |
| Very good partial response or better | 142 | (59.2) | 68 | (29.1) | <0.001 |
| Very good partial response | 96 | (40.0) | 47 | (20.1) | |
| Partial response | 57 | (23.8) | 80 | (34.2) | |
| Minimal response | 10 | (4.2) | 20 | (8.5) | |
| Stable disease | 24 | (10.0) | 47 | (20.1) | |
| Progressive disease | 5 | (2.1) | 16 | (6.8) | |
| Response could not be evaluated | 2 | (0.8) | 3 | (1.3) | |

P values were calculated with the use of the Cochran-Mantel-Haenszel chi-square test.
Criteria for a stringent complete response include the criteria for a complete response plus a normal free light-chain ratio and absence of clonal plasma cells as assessed by immunohistochemical or immunofluorescence analysis or by two-color-to-four-color flow cytometry.

### Safety

Most patients in the DVd and the Vd had at least one adverse event after the start of treatment (98.8% and 95.4%, respectively). Higher rates of grade 3 or 4 adverse events were observed in the DVd than in the Vd (76.1% vs. 62.4%). Three of the most common grade 3 or 4 adverse events reported in the DVd and the Vd were thrombocytopenia (45.3% and 32.9%, respectively), anemia (14.4% and 16.0%, respectively), and neutropenia (12.8% and 4.2%, respectively).

The percentage of patients who discontinued treatment because of at least one adverse event was similar in the DVd and the Vd (7.4% and 9.3%, respectively). The most common adverse events (occurring in at least 1% of patients in either group) that led to treatment discontinuation were peripheral sensory neuropathy (0.4% and 2.5%, respectively) and pneumonia (1.2% and 0.4%, respectively). Adverse events that led to death were reported in 13 patients (5.3%) in the DVd and in 14 patients (5.9%) in the Vd; these events were mainly a result of the general deterioration of the patients' physical health (0.4% and 1.3%, respectively). Other adverse events leading to death that were reported in 2 or more patients in either treatment group were pneumonia (1 patient in the DVd and 2 in the Vd), ischemic stroke (2 patients and no patients, respectively), and respiratory failure (2 patients and no patients, respectively). No cases of immunogenicity were reported in the DVd, and no cases of hemolysis were reported in either treatment group. Infusion-related reactions of any grade that were associated with daratumumab were reported in 45.3% of the patients; for 98.2% of these patients, the events occurred during the first infusion. Infusion-related reactions were mostly limited to grade 1 or 2 events; at least one grade 3 event was reported in 21 patients (8.6%), and no grade 4 events were reported. The most common adverse event terms that were documented by the investigator as infusion-related reactions were dyspnea (10.7%), bronchospasm (9.1%), and cough (7.0%). Two patients discontinued treatment because of infusion-related reactions: bronchospasm in 1 patient and bronchospasm, laryngeal edema, and rash in the other patient.

### Example 2. Study Design NCT02076009 (Pollux)

The purpose of this study was to assess the effects of administration of daratumumab when combined with lenalinomide and dexamethasone compared with lenalinomide and dexamethasone alone, for participants with relapsed or refractory multiple myeloma. The study was a multicenter, randomized open-label active-controlled phase 3 trial. A prespecified interim analysis has been described in Dimopoulos et al., NEJM 375: 1319-31, 2016. The clinical trial number for this study is NCT02076009.

### Eligibility

Patients with documented progressive multiple myeloma (according to International Myeloma Working Group IMWG criteria) who had received at least one previous therapy for multiple myeloma and had at least a partial response to the at least previous therapy were eligible.

### Exclusion Criteria

Patients who had received daratumumab or other anti-CD38 therapies previously, patients who were refractory to or who had unacceptable side effects from lenalinomide, patients with a neutrophil count<1000 cells/mm³, hemoglobin<7.5 g/dl, platelet count<75,000/mm³, creatinine clearance<20 ml/min per 1.73 m² body-surface area, alanine aminotransferase or aspartate aminotransferase level of 2.5 or more times the upper limit of the normal range, and bilirubin level of 1.5 or more times the upper limit of the normal range, or creatinine clearance of less than 30 ml per minute were excluded.

### Trial Treatments

Patients were randomly assigned in a 1:1 ratio to receive daratumumab, lenalinomide and dexamethasone (DRd; “daratumumab group”) or lenalinomide and dexamethasone (Rd; “control group”). Randomization was stratified by International Staging System (ISS), number of prior treatment programs (1 vs. 2 or 3 vs. >3), and prior lenalinomide treatment (“no” vs. “yes”).

Daratumumab was administered as an IV infusion at a dose of 16 mg/kg weekly (on days 1, 8, 15, and 22) for 8 weeks during cycles 1 and 2, every 2 weeks (on days 1 and 15) for 16 weeks (cycles 3 through 6), and every 4 weeks thereafter. Both groups received lenalidomide at a dose of 25 mg orally on days 1 to 21 of each cycle if the creatinine clearance was more than 60 ml per minute (or a dose of 10 mg daily if the creatinine clearance was 30 to 60 ml per minute) and dexamethasone at a dose of 40 mg weekly. For the DRd, the dose of dexamethasone was split: dexamethasone was administered at a dose of 20 mg before infusion as prophylaxis for infusion-related reactions and 20 mg was administered the next day.

### Primary Outcome Measures

Progression-free survival (PFS), defined as the time from the date of randomization to the date of disease progression or death, whichever occurred first.

### Secondary Outcome Measures

Time to disease progression (TTP), the overall response rate (ORR), the proportion of patients with very good partial response (VGPR) or better, the duration of response, the time to response and overall survival (OS). TTP is defined as the time from the date of randomization to the date of first documented evidence of progression, as defined in the International Myeloma Working Group (IMWG) criteria. The Overall Response is defined a stringent complete response (sCR), complete response, very good partial response (VGPR) or partial response (PR) as per IMWG Criteria. Duration of response will be calculated from the date of initial documentation of a response to the date of first documented evidence of progressive disease, as defined in the IMWG criteria. Time to response is defined as the time from the date of first dose of study treatment to the date of the first documentation of observed response. VGPR is defined as a greater than 90% reduction in blood myeloma protein (M-protein) plus urine myeloma protein less than 100 mg per 24 hours. OS will be measured from the date of randomization to the date of the participant's death.

In addition, the percentage of participants with Minimal Residual Disease (MRD) will be assessed, in participants who achieve ≥VGPR by analyzing bone marrow aspiration specimens.

### Statistics

A group sequential design with one prespecified interim analysis was used to evaluate the primary end point. The O'Brien-Fleming stopping boundary at the time of the interim analysis for the primary end point was calculated with the use of a Lan-DeMets alpha-spending function on the basis of the number of events observed at the data-cutoff date. Efficacy analyses were based on the intention-to-treat population, including all patients who underwent randomization. The secondary end points were compared between the DRd and the Rd with the use of a stratified lorank test. Hazard ratios and corresponding 95% confidence intervals were estimated with the use of a stratified Cox regression model, with treatment as the sole explanatory variable. The Kaplan-Meier method was used to estimate the distribution. A stratified Cochran-Mantel-Haenszel chi-square test was used to test between-group differences.

### Interim Results at Data-Cutoff Mar. 7, 2016

At the time of the data-cutoff date, 286 patients in the DRd and 283 patients in the Rd had received at least one dose of trial treatment. 66 patients in the DRd and 132 in the Rd had discontinued treatment, mainly because of progressive disease or adverse events. Patient demographic, disease and clinical characteristics in the intention-to-treat patients in the two groups are shown in Table 3. International Staging System (ISS) disease staging was derived on the basis of the combination of serum β2-microglobulin and albumin. The ISS consists of three stages: stage I, serum β2-microglobulin level lower than 3.5 mg per liter (300 nmol per liter) and albumin level of 3.5 g per deciliter or higher; stage II, neither stage I nor III; and stage III, serum β2-microglobulin of 5.5 mg per liter or higher (470 nmol per liter). Higher stages indicate more severe disease.

TABLE 3

| | DRd | | Rd | |
|---|---|---|---|---|
| Characteristic | (N = 286) | | (N = 283) | |
| Age | | | | |
| Median (range)-Yr | 65 | (34-89) | 65 | (42-87) |
| Distribution-no. (%) | | | | |
| <65 yr | 133 | (46.5) | 140 | (49.5) |
| 65-74 yr | 124 | (43.4) | 108 | (38.2) |
| ≥75 yr | 29 | (10.1) | 35 | (12.4) |
| Race-no. (%)† | | | | |
| White | 207 | (72.4) | 186 | (65.7) |
| Black | 5 | (1.7) | 11 | (3.9) |
| Asian | 54 | (18.9) | 46 | (16.3) |
| Other or unreported | 20 | (7.0) | 40 | (14.1) |
| ECOG performance-status score-no. (%)‡ | | | | |
| 0 | 139 | (48.6) | 150 | (53.0) |
| 1 or 2 | 147 | (51.4) | 133 | (47.0) |
| ISS disease staging-no. (%) | | | | |
| I | 137 | (47.9) | 140 | (49.5) |
| II | 93 | (32.5) | 86 | (30.4) |
| III | 56 | (19.6) | 57 | (20.1) |
| Cytogenetic profile-no. (%) | | | | |
| Standard-risk cytogenetic abnormality | 193/228 | (84.6) | 176/211 | (83.4) |
| High-risk cytogenetic abnormality | 35/228 | (15.4) | 35/211 | (16.6) |
| Median time since initial diagnosis of | 3.5 | (0.4-27.0) | 4.0 | (0.4-21.7) |
| multiple myeloma (range)-yr | | | | |
| Median no. of previous lines of therapy (range) | 1 | (1-11) | 1 | (1-8) |
| Previous therapy-no. (%) | | | | |
| Autologous stem-cell transplant | 180 | (62.9) | 180 | (63.6) |
| Proteasome inhibitor | 245 | (85.7) | 242 | (85.5) |
| Immunomodulatory drug | 158 | (55.2) | 156 | (55.1) |
| Glucocorticoid | 280 | (97.9) | 281 | (99.3) |
| Alkylating agent | 268 | (93.7) | 270 | (95.4) |
| Proteasome inhibitor and immunomodulatory drug | 125 | (43.7) | 125 | (44.2) |
| Proteasome inhibitor, immunomodulatory drug, and | 118 | (41.3) | 121 | (42.8) |
| alkylating agent | | | | |
| Bortezomib and lenalidomide | 44 | (15.4) | 43 | (15.2) |
| Refractory disease-no. (%) | | | | |
| To last line of therapy | 80 | (28.0) | 76 | (26.9) |
| To proteasome inhibitor only | 57 | (19.9) | 46 | (16.3) |
| To immunomodulatory drug only | 10 | (3.5) | 11 | (3.9) |
| To proteasome inhibitor and immunomodulatory drug | 7 | (2.4) | 14 | (4.9) |

Eastern Cooperative Oncology Group (ECOG) performance status is scored on a scale from 0 to 5, with 0 indicating no symptoms and higher scores indicating increasing disability

### Efficacy

At a median follow-up of 13.5 months, a total of 169 events of disease progression or death (in 53 patients [18.5%] in the DRd vs. 116 [41.0%] in the Rd) were reported. The hazard ratio for disease progression or death in the DRd versus the Rd was 0.37 (95% confidence interval [CI], 0.27 to 0.52; P<0.001 by stratified log-rank test). The Kaplan-Meier rate of progression-free survival at 12 months was 83.2% (95% CI, 78.3 to 87.2) in the DRd and 60.1% (95% CI, 54.0 to 65.7) in the Rd. The median progression-free survival was not reached (95% CI; could not be estimated) in the DRd, as compared with 18.4 months (95% CI, 13.9 to could not be estimated) in the Rd. Similarly, in the time-to-event analysis of disease progression, a total of 148 events (in 44 patients [15.4%] in the DRd vs. 104 [36.7%] in the Rd) were observed (hazard ratio, 0.34; 95% CI, 0.23 to 0.48; P<0.001). The rate of progression-free survival at 12 months was 85.7% (95% CI, 80.9 to 89.4) in the DRd, as compared with 63.2% (95% CI, 57.1 to 68.8) in the Rd. Table 4 shows the summary of responses among patients who could be evaluated for response.

TABLE 4

| | DRd | | Rd | | P |
|---|---|---|---|---|---|
| Response Category | (N = 281) | | (N = 276) | | Value |
| Overall response | | | | | |
| No. with response | 261 | | 211 | | |
| Rate-% (95% CI) | 92.9 | (89.2-95.6) | 76.4 | (71.0-81.3) | <0.001 |
| Clinical benefit-no. (%) | 266 | (94.7) | 237 | (85.9) | |
| Best overall | | | | | |
| response-no. (%) | | | | | |
| Complete response or better | 121 | (43.1) | 53 | (19.2) | <0.001 |
| Stringent complete response | 51 | (18.1) | 20 | (7.2) | |
| Complete response | 70 | (24.9) | 33 | (12.0) | |
| Very good partial | 213 | (75.8) | 122 | (44.2) | <0.001 |
| response or better | | | | | |
| Very good partial response | 92 | (32.7) | 69 | (25.0) | |
| Partial response | 48 | (17.1) | 89 | (32.2) | |
| Minimal response | 5 | (1.8) | 26 | (9.4) | |
| Stable disease | 13 | (4.6) | 33 | (12.0) | |
| Progressive disease | 0 | | 4 | (1.4) | |
| Response could not | 2 | (0.7) | 2 | (0.7) | |
| be evaluated | | | | | |

P values were calculated with the use of the Cochran-Mantel-Haenszel chi-square test.
Criteria for a stringent complete response include the criteria for a complete response plus a normal free light-chain ratio and absence of clonal plasma cells as assessed by immunohistochemical or immunofluorescence analysis or by two color-to-four-color flow cytometry.

### Safety

The most common adverse events of any grade during treatment (in >15% of the patients in either group) and adverse events of grade 3 or 4 (in >5% of the patients in either group) for the safety population were neutropenia, anemia, thrombocytopenia, febrile neutropenia, lymphopenia, diarrhea, fatigue, upper respiratory tract infection, constipation, cough, muscle spasms, nasopharyngitis, nausea, pyrexia, insomnia, dyspnea, back pain, vomiting, asthenia, peripheral edema, and pneumonia. Adverse events that occurred at a frequency of 10% or more in the DRd versus the Rd were neutropenia, diarrhea, upper respiratory tract infection, and cough, most of which resulted from longer exposure to treatment in the DRd. Deep-vein thrombosis was reported in 1.8% of the patients in the DRd and in 3.9% of those in the Rd. In the DRd, 51.9% of patients had neutropenia of grade 3 or 4, as compared with 37.0% of those in the Rd; thrombocytopenia of grade 3 or 4 occurred in 12.7% and 13.5% of the patients, respectively. The percentage of patients with adverse events leading to the discontinuation of treatment was similar in the two groups: 6.7% in the DRd and 7.8% in the Rd. Adverse events leading to death occurred in 11 patients (3.9%) in the DRd and in 15 (5.3%) in the Rd. The most common adverse events leading to death were acute kidney injury (in 0.4% of the patients in the DRd and in 1.1% in the Rd), septic shock (in 1.1% and 0.4%, respectively), and pneumonia (in 0.7% in each group).

The incidence of daratumumab infusion-related reactions of any grade was 47.7%, with 92% of the reactions occurring during the first infusion. These reactions were mostly of grade 1 or 2; a total of 15 patients (5.3%) had grade 3 infusion reactions, and no patient had an event of grade 4 or 5. The most common infusion-related reactions were cough (in 8.5% of the patients), dyspnea (in 8.5%), and vomiting (in 5.7%). One patient discontinued daratumumab because of a grade 3 infusion-related event, recovered, and continued to receive lenalidomide and dexamethasone treatment.

### Example 3. Efficacy of Daratumumab in Combination with Lenalidomide and Dexamethasone or Bortezomib and Dexamethasone in Relapsed or Refractory Multiple Myeloma Patients (RRMM) in High-Risk Patients

### Methods

The analysis sets included subgroup analyses of patients from the POLLUX (Example 2) and CASTOR (Example 1) trials who had received 1 to 3 prior lines of therapy (1-3 PL subgroup). Cytogenetic abnormalities were determined at the screening visit prior to randomization by fluorescence in-situ hybridization (FISH) based on local laboratory assessment. Patients with high-risk cytogenetics included those who had one or more of the following abnormalities: t(4;14)(p16;q32), t(14;16)(q32;q23), or del17p; standard-risk patients were defined as those who underwent cytogenetic testing and did not meet the high-risk criteria.

### Results: Pollux

In the 1-3 PL subgroup (DRd, n=272; Rd, n=264), PFS was significantly improved with DRd vs Rd (median: not reached [NR] vs 18.4 months; HR, 0.36; 95% CI, 0.25-0.50; P<0.0001), with estimated 12-month PFS rates of 83.2% vs 6.4%, respectively. Time to progression was also significantly longer with DRd vs Rd (median: NR vs 18.4 months; HR, 0.32; 95% CI, 0.22-0.46; P<0.0001). ORR (94% vs 77%), rates of very good partial response (VGPR) or better (76% vs 45%) and complete response (CR) or better (44% vs 20%) were significantly higher with DRd vs Rd, respectively (P<0.0001 for all). Among responders, median time to VGPR or better was 2.8 months in DRd vs 2.9 months in Rd; median time to CR or better was 6.7 months vs 7.5 months, respectively.

For pts in the 1-3 PL subgroup with high-risk cytogenetics (n=33 in each treatment group), significantly longer PFS was observed in DRd vs Rd (median: NR vs 8.3 months; HR, 0.30; 95% CI, 0.14-0.67; P=0.0019). Significantly higher ORR (91% vs 69%; P=0.0267), rate of VGPR or better (73% vs 28%; P=0.0004), and rate of CR or better (36% vs 9%; P=0.0104) were achieved in pts with high-risk cytogenetic status treated with DRd vs Rd, respectively.

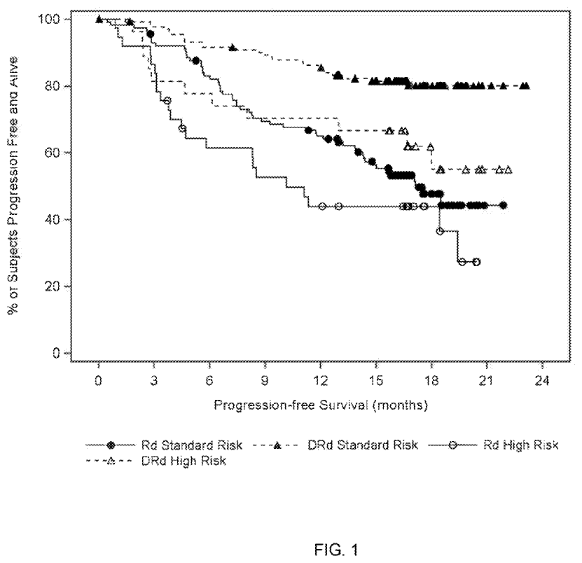

FIG. 1 shows the percentage subjects who are progression free and alive in each subgroup over time.

### Results: Castor

Median follow-up was 7.4 months. In the 1 to 3 prior lines (1-3 PL) subgroup (DVd, n=229; Vd, n=219), PFS was significantly longer with DVd vs Vd (median: not reached [NR] vs 7.3 mo; HR, 0.39; 95% CI, 0.28-0.55; P<0.0001); estimated 12-month PFS rates were 62.2% vs 29.3%, respectively. Median time to progression (TTP) among 1-3 PL pts was NR vs 7.4 months, respectively (HR, 0.29; 95% CI, 0.20-0.43; P<0.0001). Overall response rate (ORR) was significantly higher with DVd vs Vd (84% vs 67%; P<0.0001) and was associated with higher rates of very good partial response (VGPR) or better (62% vs 32%; P<0.0001).

Among 1-3 PL pts with standard-risk cytogenetic status, PFS was significantly prolonged in DVd vs Vd (HR, 0.38; 95% CI, 0.25-0.58; P<0.0001), and estimated 12-month PFS rates were 58.7% vs 27.0%, respectively. PFS was also significantly longer in pts with high-risk cytogenetics who received DVd vs Vd (HR, 0.46; 95% CI, 0.22-0.97; P=0.0367) with estimated 12-month PFS rates of 63.2% vs 26.7%, respectively. Lastly, the rate of MRD-negative patients was significantly higher (4 fold or greater) at all evaluated thresholds (10⁻⁴, 10⁻⁵, and 10⁻⁶) among the 1-3 PL subgroup.

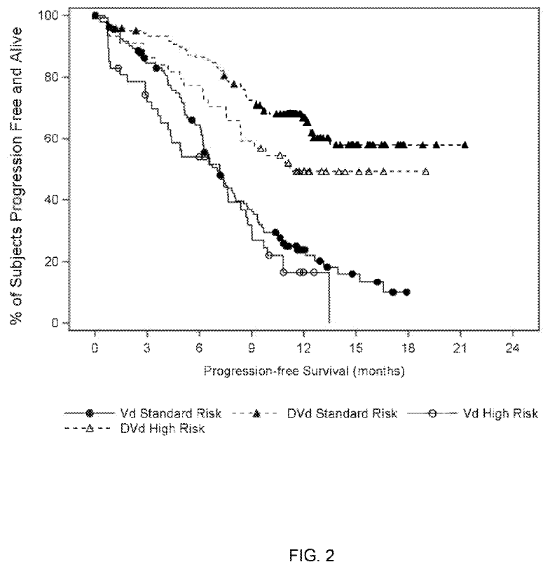

FIG. 2 shows the percentage subjects who are progression free and alive in each subgroup over time.

### CONCLUSION

The addition of DARA to Rd significantly improved outcomes in RRMM patients with high-risk cytogenetics, and the addition of DARA to Vd showed encouraging trends towards improved PFS and response rates in these pts. Remarkably, outcomes by PFS in high risk patients treated with DRd were at least comparable to those for standard-risk patients treated with Rd alone. These results suggest that targeting CD38 in combination with Rd may help overcome the poor outcomes associated with high-risk cytogenetic status.

### Example 4. MRD Analysis

### MRD Sample Collection and Processing

In Study POLLUX, MRD status was assessed (blinded to treatment cohort) at the time of suspected CR, and at 3 and 6 months post-suspected CR for subjects who maintained this response. In Study CASTOR, MRD was evaluated (blinded to treatment cohort) at the time of suspected CR and for subjects who reached MRD negativity, additional analysis was done at the end of Vd background therapy (6 months after study therapy began) and finally 6 months after the end of Vd background therapy (12 months after study therapy began). The MRD assay was performed using bone marrow aspirates (BMA) and evaluated by the ClonoSEQ™ assay (Adaptive Biotechnologies, Seattle, WA, USA). Briefly, bone marrow mononuclear cells were isolated from 2-3 mL of BMA by lymphoprep (Ficoll) separation at Covance Central Laboratory Services (Indianapolis, IN, U.S.A; Geneva, Switzerland; Singapore) within 48 hours of collection. The cells were stored as a dry pellet at −70° C. An important note is that lymphoprep separation typically removes >95% of the granulocytes in the sample, whereas these cells are retained when the sample is processed by red blood cell (RBC) lysis. In normal bone marrow, granulocytes account for 25-50% of the cell fraction and lymphoprep separation will disproportionately reduce the total cell number in the sample analyzed. This may lead to differences in MRD negativity rates in other studies using RBC lysis since these studies have a 2-fold greater denominator and will be less stringent than the MRD determinations in Studies POLLUX and CASTOR.

Genomic DNA was isolated and amplified using a set of multiplexed, locus-specific primer sets for the IGH complete (IGH-VDJH), IGH incomplete (IGH-DJH), and immunoglobulin x locus (IGK). The amplified product was subjected to sequencing, and the sequences and frequencies of the different clonotypes in the sample were obtained. To define myeloma clones for gene rearrangements in samples obtained at diagnosis, a frequency threshold of 5% (i.e., any clonotype present at a frequency of >5% was regarded as originating from the myeloma clone) was applied. MRD was assessed in the clinical samples with a high-frequency myeloma clone using the IGH-VDJH, IGH-DJH, and/or IGK assays as previously described (Vij R, Mazumder A, Klinger M, et al. Deep sequencing reveals myeloma cells in peripheral blood in majority of multiple myeloma patients. Clin Lymphoma Myeloma Leuk. 2014; 14:131-139). The myeloma-derived sequences identified at diagnosis were used as a target to assess the presence of MRD in the follow-up samples for each subject analyzed. For MRD quantitation, multiple sequencing reads were assessed for each rearranged B cell in the reaction. Once the absolute amount of total cancer-derived molecules present in a sample was determined, a final MRD measurement was calculated, providing the number of cancer-derived molecules per 1 million cell equivalents. In cases where two or more tumor clones existed, the clone with the highest MRD value was reported.

### Methods for Data Analysis

### Clinical Data

The input dataset from the clinical cutoff date for Study POLLUX and for Study CASTOR were used for this analysis. All the data analysis and generation of relevant graphs were performed exclusively using R software. The analysis population was the ITT population, which included all randomized subjects. Best response reported in this analysis was the best confirmed response by computerized algorithm in accordance to IMWG response and disease progression criteria.

### MRD Data

### MRD Data Technical Aspects

A baseline diagnostic sample from each subject was used to characterize the myeloma clone when present at a frequency of >5%. A sample failed to calibrate if a high-frequency myeloma clone could not be identified. Calibration rates of 75% and 77% in Studies POLLUX and CASTOR respectively were observed for the MRD assays using the BMA samples collected at Screening in these MM subjects (Table 5). The fact that these samples are from relapsed or refractory MM subjects could contribute to the higher calibration rates than those observed with newly diagnosed MM samples. Additionally, as these studies represent the first, prospective evaluation of MRD in a global study in relapsed or refractory MM subjects, this calibration rate may be reflective of a real world application of the technology versus in an academic research setting.

TABLE 5

| | Calibrates | | Fails to calibrate | | |
|---|---|---|---|---|---|
| Study | n (%) | | n (%) | | Total |
| POLLUX | 146 | 75.26 | 48 | 24.74 | 194 |
| CASTOR | 92 | 77.31 | 27 | 22.69 | 119 |

MRD status was to be determined at sensitivity thresholds of 10⁻⁴, 10⁻⁵, and 10⁻⁶. Importantly, a stringent criterion of cellular input equivalents of at least 10,000, 100,000, and 1,000,000, respectively, is required for determination of MRD status at each threshold. For a subset of the samples in both Studies POLLUX and CASTOR, the number of input cells did not reach the required threshold of 10⁻⁵ or 10⁻⁶ and MRD calls were therefore defined as “MRD ambiguous” and were counted as MRD positive in those samples at the threshold being evaluated (Table 6).

### MRD Data Handling

The MRD status calls, total input cell equivalent, and clone count data were obtained from the clinical cut off dates. The MRD data on the individual receptors, containing the total input cell equivalent, clone count data, as well the clone frequency was provided by the vendor (Adaptive Biotechnologies) and stored in Cyberlab data repository.

Clone count and frequency provided by the vendor were calculated as:

$$Clone count = \frac{\begin{matrix}( 1 / sensitivity threshold ) * Total input cance r double \\ stranded molecules in reaction of Follow - Up Sample\end{matrix}}{\begin{matrix}Total input cell equivalent in \\ Follow - Up Sample for this receptor\end{matrix}}$$

where the numerator represents the cancer B cell number, and the denominator the total cell number (=cancer B cells+B cells+non-B cells).

$$Frequency = \frac{\begin{matrix}Total input cance r double stranded \\ molecules in reaction of Follow - Up Sample\end{matrix}}{\begin{matrix}Total input rearranged receptor double \\ stranded molecules in Follow - Up Sample\end{matrix}} * 100.$$

where the numerator represents the cancer B cell number, and the denominator the total B cell number (=cancer B cells+B cells).

Retested sample data were summarized by averaging the frequency and summing the clone count as per instructions by the vendor.

For each MRD assessment sample, an MRD status call was determined that was either “MRD NEGATIVE,” “MRD POSITIVE,” or “MRD AMBIGUOUS,” for each of three different sample detection limit thresholds, 10⁻⁴, 10⁻⁵ or 10⁻⁶. An “MRD NEGATIVE” test result was obtained if the number of clones detected was <1, and the number of input cells was >the detection limit threshold (one of 10⁻⁴, 10⁻⁵ or 10⁻⁶). An “MRD POSITIVE” test result was obtained if the number of clones detected was >1 or in the ITT population without MRD assessment. An “MRD AMBIGUOUS” test result was obtained if the number of clones detected was <1, but the total input cell equivalent did not reach the required sensitivity level (of either 10⁻⁴, 10⁻⁵ or 10⁻⁶).

When comparing MRD negative counts between subgroups, MRD calls were dichotomized into MRD negative or MRD positive, where MRD positive included subjects who were tested and found positive at all time points or ambiguous or were not tested.

TABLE 6

| | | 10⁻⁴, n | 10⁻⁵, n | 10⁻⁶, n |
|---|---|---|---|---|
| POLLUX | MRD Negative | 153 | 116 | 48 |
| DRd | MRD Positive | 41 | 77 | 121 |
| | MRD Ambiguous | 0 | 1 | 25 |
| POLLUX | MRD Negative | 30 | 15 | 6 |
| Rd | MRD Positive | 26 | 38 | 45 |
| | MRD Ambiguous | 0 | 3 | 5 |
| CASTOR | MRD Negative | 38 | 20 | 10 |
| DVd | MRD Positive | 29 | 46 | 55 |
| | MRD Ambiguous | 0 | 1 | 2 |
| CASTOR | MRD Negative | 9 | 6 | 2 |
| Vd | MRD Positive | 27 | 30 | 34 |
| | MRD Ambiguous | 0 | 0 | 0 |

### Results

### Primary MRD Results

Briefly, for the ITT population, the DRd group demonstrated a greater incidence of MRD negativity compared with the Rd group. Twenty-nine percent (29%) of the subjects in the DRd group achieved MRD negativity status at the threshold of 10⁻⁴ versus 7.8% in the Rd group (Mantel-Haenszel Odds Ratio=4.88; 95% CI: 2.94, 8.08; p=<0.0001). Similarly, for Study CASTOR, subjects treated with DVd demonstrated a greater incidence of MRD negativity (14%) compared with 3% of subjects treated with Vd (Mantel-Haenszel Odds Ratio Estimate=5.56; 95% CI: 2.37, 13.04; p=<0.0001). As an exploratory analysis, MRD rates were also evaluated at two more stringent thresholds, 10⁻⁵ and 10⁻⁶. In Study POLLUX, the MRD negativity rate was significantly higher for subjects in the DRd group compared with subjects in the Rd at both lower thresholds. In Study CASTOR, DVd had increased MRD negative rates at both lower thresholds compared to Vd, but was significant only at 10⁻⁵ threshold.

### Overall Best Confirmed Response Call and MRD Status

A statistically significant improvement in responses were observed for subjects treated with DRd compared with those treated with Rd in Study POLLUX, and for subjects treated with DVd compared with those treated with Vd in Study CASTOR.

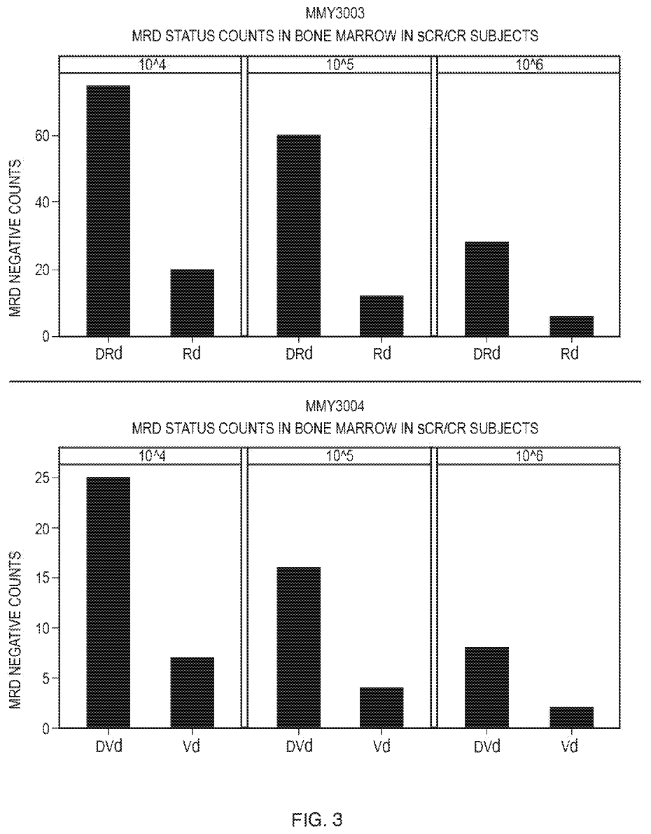

In addition to a higher ORR in the daratumumab combination groups in Studies POLLUX and CASTOR (data not shown), a higher incidence of MRD negative status was detected in the daratumumab combination groups in both studies (FIG. 3). In Study POLLUX, 75 subjects (26% of ITT) with a best confirmed clinical response of sCR or CR (38 and 37 subjects, respectively) reached MRD negative status at a threshold of 10⁻⁴ with DRd treatment. In the Rd treatment group, 20 subjects (7% of ITT) with a best confirmed clinical response of sCR or CR (10 subjects of each) reached MRD negativity at 10⁻⁴ (Table 7).

In Study CASTOR, 25 subjects (10% of ITT) with a best confirmed clinical response of sCR or CR (5 and 20 respectively) reached MRD negative status at 10⁻⁴ with DVd treatment. With Vd treatment, MRD negativity at 10⁻⁴ threshold was observed in 7 subjects (3% of ITT) with a best confirmed clinical response of sCR or CR (3 and 4 subjects, respectively) (Table 7). At 10⁻⁵ and 10⁻⁶ sensitivity thresholds in both studies, the MRD negative counts decreased. Daratumumab containing regimens consistently showed 3-fold or greater increases in MRD negativity rate compared with the control groups regardless of the background therapy.

TABLE 7

| | | DRd | Rd | | | | |
|---|---|---|---|---|---|---|---|
| | | SCR/ | (sCR/ | DRd | Rd | DRd | Rd |
| | MRD | (CR) | CR) | (sCR) | (sCR) | (CR) | (CR) |
| MMY | 10-4 | 75 | 20 | 38 | 10 | 37 | 10 |
| 3003 | | (26.2%) | (7.1%) | (13%) | (3.5%) | (12.9%) | (3.5%) |
| (POL- | 10-5 | 60 | 12 | 31 | 4 | 29 | 8 |
| LUX) | | (21%) | (4.2%) | (11%) | (1.4%) | (10.1%) | (2.8%) |
| | 10-6 | 28 | 6 | 20 | 2 | 8 | 4 |
| | | (9.8%) | (2.1%) | (7%) | (0.7%) | (2.8%) | (1.4%) |
| | | DVd | Vd | | | | |
| | | (sCR/ | (sCR/ | DVd | Vd | DVd | Vd |
| | MRD | CR) | CR) | (sCR) | (sCR) | (CR) | (CR) |
| MMY | 10-4 | 25 | 7 | 5 | 3 | 20 | 4 |
| (CAS- | | (10%) | (2.83%) | (2%) | (1.2%) | (8%) | (1.6%) |
| TOR) | 10-5 | 16 | 4 | 2 | 3 | 14 | 1 |
| 3004 | | (6.4%) | (1.62%) | (0.8%) | (1.2%) | (5.6%) | (0.4%) |
| | 10-6 | 8 | 2 | 1 | 1 | 7 | 1 |
| | | (3.2%) | (0.81%) | (0.4%) | (0.4%) | (2.8%) | (0.4%) |

### Minimal Residual Disease Over Time

In Study POLLUX, MRD assessment was performed (blinded to treatment cohort) at the time of suspected CR, and at 3 and 6 months post-suspected CR for subjects who maintained this response. In Study CASTOR, MRD was evaluated for subjects at the time of suspected CR (blinded to treatment cohort) at the end of Vd background therapy (six months after study start) and 6 months after the end of Vd background therapy (12 months after study start) in the ITT population. The MRD assessment over time enabled investigation of depth and duration of MRD response in relation to the clinical response (data not shown).

As in both studies, MRD was assessed in subjects with suspected CR, the MRD negative status in these treatment groups was observed to coincide with the clinical response change to CR or sCR for majority of the subjects. Bone marrow samples from a limited number of subjects with clinical responses of partial response (PR), stable disease (SD), or minimal response (MR) were inadvertently shipped for MRD analysis which was intended for subjects with a response of ≥VGPR. As expected, these subjects tested MRD positive, with the exception of 1 sample from one subject in each of the daratumumab combination groups (DRd and DVd).

The single subject in Study POLLUX who showed MRD negative status, but best clinical response of <VGPR suffered from a baseline plasmacytoma, that achieved a maximum reduction of 36%. The best response was MR by the central algorithm despite a VGPR response evaluated by the investigator (data not shown). At lower thresholds for MRD negativity (10⁻⁵ and 10⁻⁶), this subject was MRD positive (data not shown).

Further assessment of the single subject in Study CASTOR who achieved MRD negativity status revealed that, although the index clone identified at screening was reduced to MRD negative status (81.9% to 0.4% of the bone marrow cells), an unrelated myeloma clone present at low level at screening became dominant (7%) at the time of testing. This offers an explanation as to why the subject had progressive disease as the current response by the investigator and central algorithm, yet is counted as being MRD negative at the hierarchical threshold of 10⁻⁴ (data not shown). At lower thresholds for MRD negativity (10⁻⁵ and 10⁻⁶), this subject was MRD positive (data not shown).

### MRD Status and Progression Free Survival

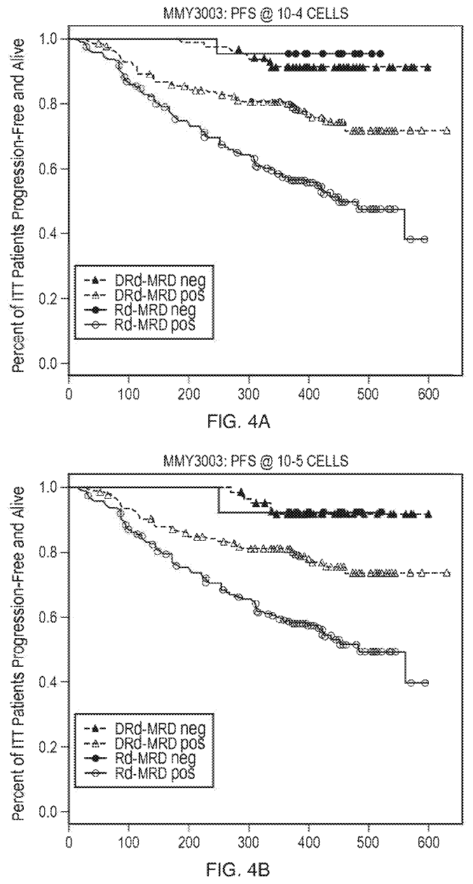

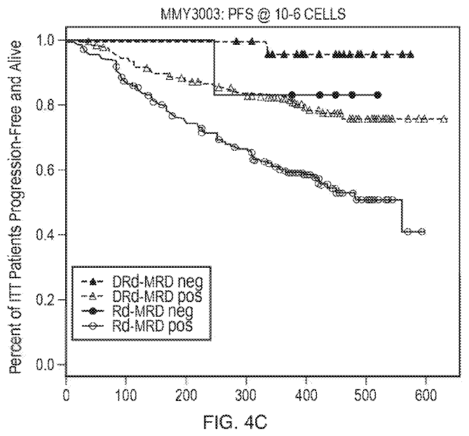

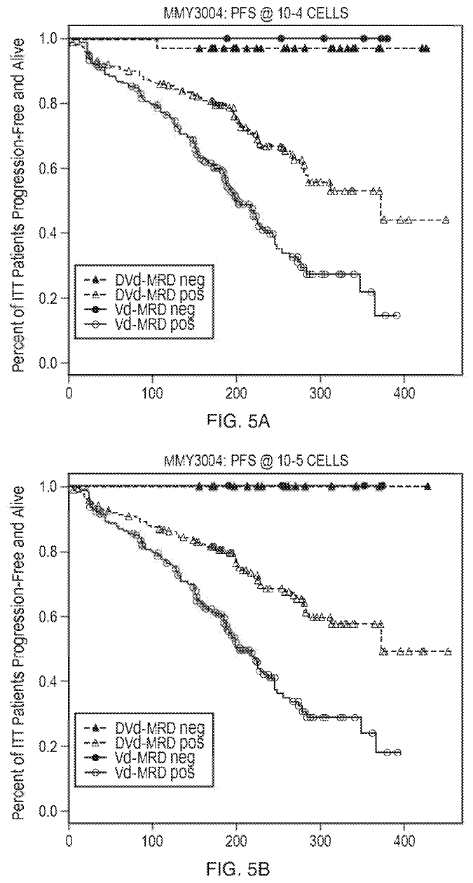

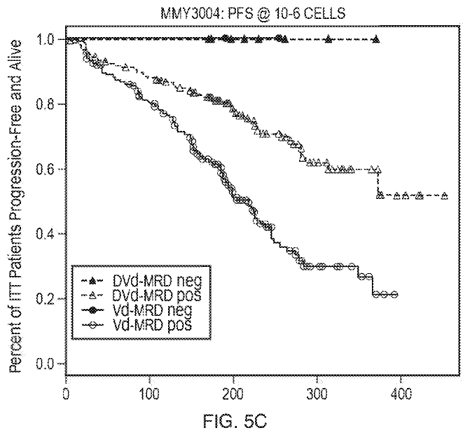

Subjects who achieved MRD negative status in either of the treatment groups experienced fewer PFS events compared with MRD positive subjects, at all three thresholds tested (FIG. 4A, FIG. 4B and FIG. 4C for POLLUX, FIG. 5A, FIG. 5B and FIG. 5C for CASTOR). Of the subjects who remain MRD positive, improved PFS was observed in the daratumumab combination group compared to standard of care regimens, Rd and Vd, in both studies.

### MRD Status and Prior Lines of Therapy

In Study POLLUX, at 10⁻⁴, MRD negative status was reached in subjects treated with DRd with up to 4 prior lines of therapy, with the highest rate in subjects who received one prior line (n=41, 14.3% ITT, Table 8). With Rd treatment, MRD negative status was reached at a lower rate for each of the prior lines, with subjects receiving 1 or 2 prior lines and only 2 subjects (0.7% ITT) receiving 4+ prior lines of therapy. No subjects with 3 prior lines of therapy reached MRD negative status. A similar pattern was observed for detection thresholds 10⁻⁵ and 10⁻⁶.

In Study CASTOR, at 10⁻⁴, the highest MRD negative rate was reached with DVd treatment (n=20, 8%). Subjects with 2 or 3 prior lines of therapy showed a lower MRD negativity rate (n=10, 4% and n=4, 1.6% of ITT, respectively). In the Vd treatment group, only subjects with 1 or 2 prior lines of therapy reached MRD negative status (n=4, 1.6% and 3, 1.2%, respectively). Similar trends were observed for 10⁻¹. At 10⁻⁶, no subjects with 3+ prior lines of therapy in the DVd treatment group and 2+ prior lines of therapy in the Vd treatment group reached MRD negative state.

TABLE 8

| | | Prior | | | | |
|---|---|---|---|---|---|---|
| | | lines | | | | |
| | | of | DRd- | Rd- | DRd- | Rd- |
| | | ther- | MRD | MRD | MRD | MRD |
| | MRD | apy | neg | neg | pos | pos |
| MMY | 10-4 | 1 | 41 (14.3%) | 14 (4.9%) | 108 (37.8%) | 132 (46.6%) |
| 3003 | | 2 | 32 (11.2%) | 6 (2.1%) | 53 (18.5%) | 74 (26.1%) |
| (POL- | | 3 | 8 (2.8%) | 0 (0%) | 30 (10.5%) | 38 (13.4%) |
| LUX) | | 4+ | 2 (0.7%) | 2 (0.7%) | 12 (4.2%) | 17 (6%) |
| | 10-5 | 1 | 31 (10.8%) | 9 (3.2%) | 118 (41.3%) | 137 (48.4%) |
| | | 2 | 24 (8.4%) | 4 (1.4%) | 61 (21.3%) | 76 (26.9%) |
| | | 3 | 7 (2.4%) | 0 (0%) | 31 (10.8%) | 38 (13.4%) |
| | | 4+ | 2 (0.7%) | 0 (0%) | 12 (4.2%) | 19 (6.7%) |
| | 10-6 | 1 | 12 (4.2%) | 5 (1.8%) | 137 (47.9%) | 141 (49.8%) |
| | | 2 | 12 (4.2%) | 1 (0.4%) | 73 (25.5%) | 79 (27.9%) |
| | | 3 | 2 (0.7%) | 0 (0%) | 36 (12.6%) | 38 (13.4%) |
| | | 4+ | 2 (0.7%) | 0 (0%) | 12 (4.2%) | 19 (6.7%) |
| | | Prior | | | | |
| | | lines | | | | |
| | | of | DVd- | Vd- | DVd- | Vd- |
| | | ther- | MRD | MRD | MRD | MRD |
| | MRD | apy | neg | neg | pos | pos |
| MMY | 10-4 | 1 | 20 (8%) | 4 (1.6%) | 102 (40.6%) | 109 (44.1%) |
| 3004 | | 2 | 10 (4%) | 3 (1.2%) | 60 (23.9%) | 71 (28.7%) |
| (CAS- | | 3 | 4 (1.6%) | 0 (0%) | 33 (13.1%) | 32 (13%) |
| TOR) | | 4+ | 0 (0%) | 0 (0%) | 22 (8.8%) | 28 (11.3%) |
| | 10-5 | 1 | 10 (4%) | 3 (1.2%) | 112 (44.6%) | 110 (44.5%) |
| | | 2 | 6 (2.4%) | 1 (0.4%) | 64 (25.5%) | 73 (29.6%) |
| | | 3 | 2 (0.8%) | 0 (0%) | 35 (13.9%) | 32 (13%) |
| | | 4+ | 0 (0%) | 0 (0%) | 22 (8.8%) | 28 (11.3%) |
| | 10-6 | 1 | 5 (2%) | 2 (0.8%) | 117 (46.6%) | 111 (44.9%) |
| | | 2 | 4 (1.6%) | 0 (0%) | 66 (26.3%) | 74 (30%) |
| | | 3 | 0 (0%) | 0 (0%) | 37 (14.7%) | 32 (13%) |
| | | 4+ | 0 (0%) | 0 (0%) | 22 (8.8%) | 28 (11.3%) |

### MRD Case Study Examples

A number of MRD case study examples were identified from these studies that showed that the ClonoSEQ™ assay can be used to investigate how individual tumor clone sequences behave upon treatment in individual subjects.

MRD Negative Responders with Maintained MRD Negative Status

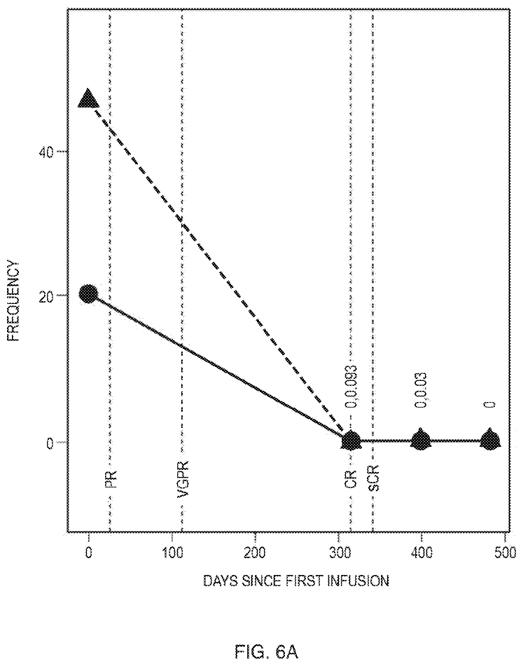

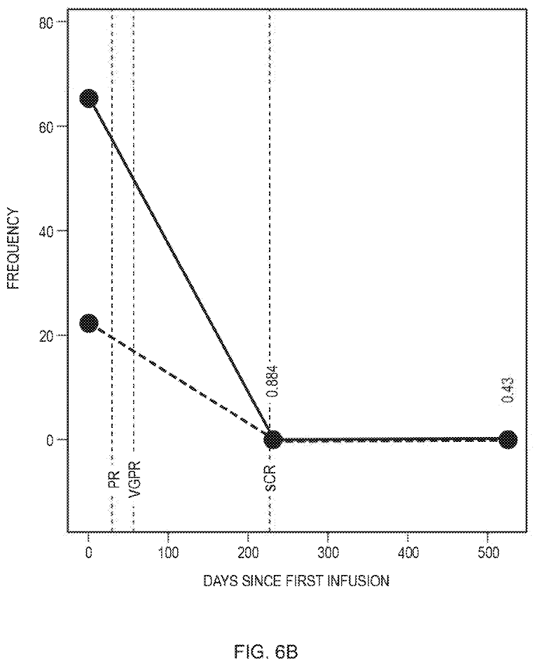

In Study POLLUX, MRD was assessed at multiple time points upon treatment, i.e., at the time of suspected CR, and at 3 and 6 months post-suspected CR for subjects who maintained this response. A large number of subjects (majority of which were in the DRd group), reached MRD negative state at a time of suspected complete response and maintained it over time. FIG. 6A and FIG. 6B show the MRD profiles of two such subjects.

### MRD Positivity in Partial and Non-Responders

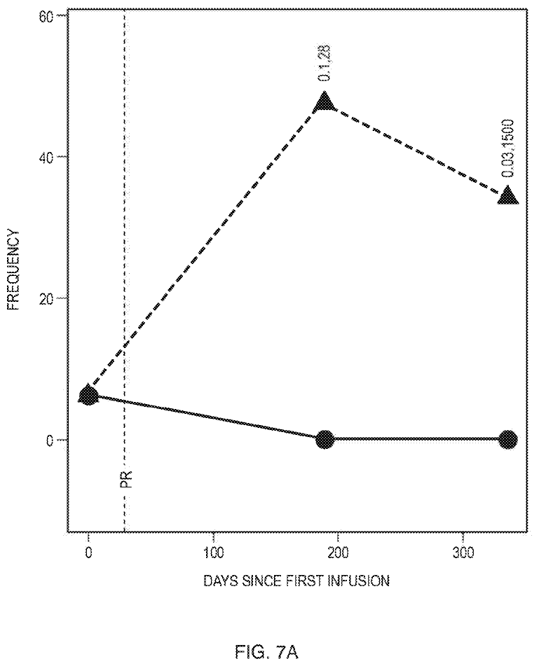

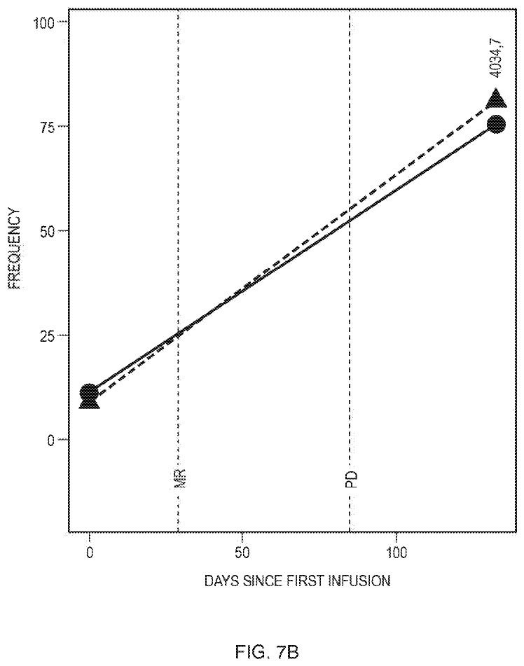

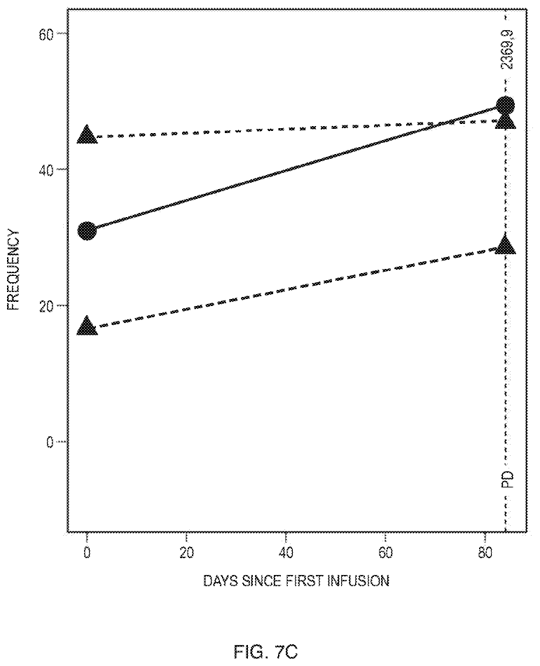

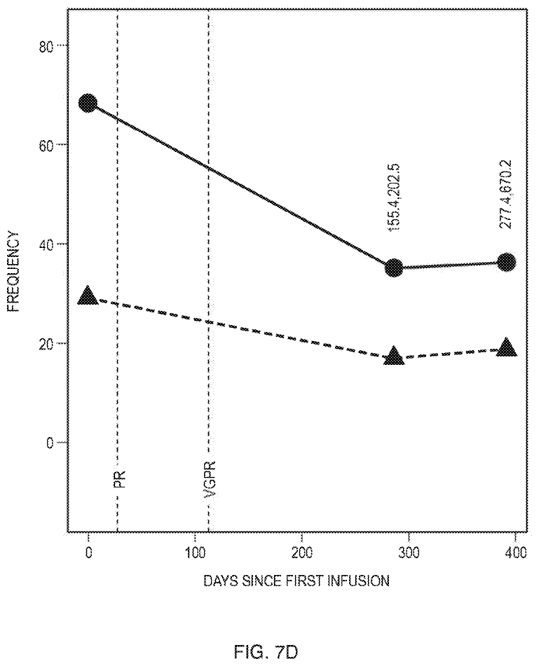

A limited number of samples of subjects with clinical responses of PR, SD, or MR were inadvertently shipped for MRD analysis, which was only intended for subjects with a response of ≥VGPR. As expected clinically, in these as well as in other subjects that can be identified, the MRD negative status was not reached and their clone receptor frequency remained high. FIG. 7A, FIG. 7B, FIG. 7C and FIG. 7D show the MRD profiles of four such subjects.

### MRD at Treatment Relapse

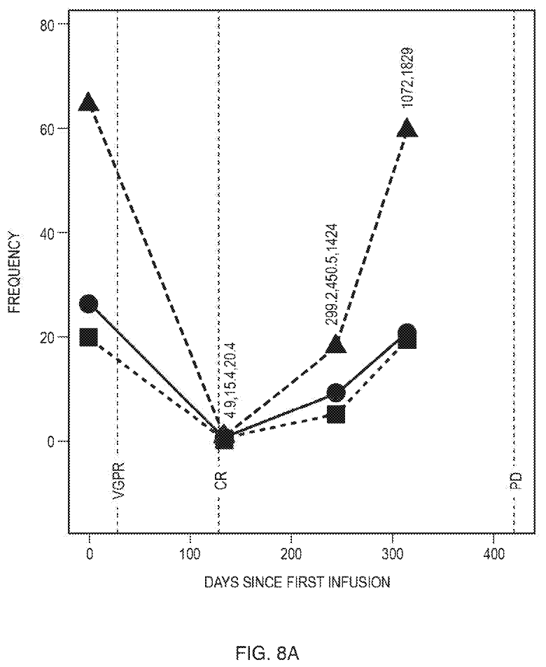

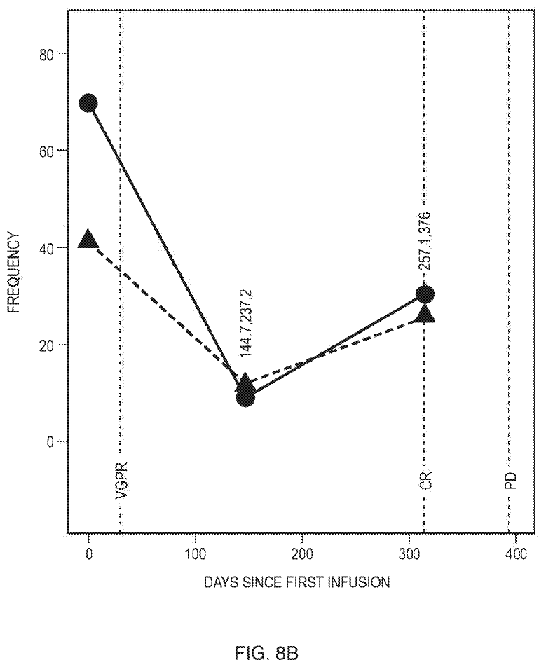

Clonal response and then expansion was observed in some subjects who initially showed clinical response, after which they experienced progressive disease. FIG. 8A and FIG. 8B show the MRD profiles of two such subjects. In these subjects, the MRD diagnostic clone was decreased at time of CR, after which the clone frequency increased, and progressive disease was later clinically detected. These cases suggest that MRD testing could be used as a sensitive measure of disease response, including an early sign of relapse and progression.

### Response Depth Increases

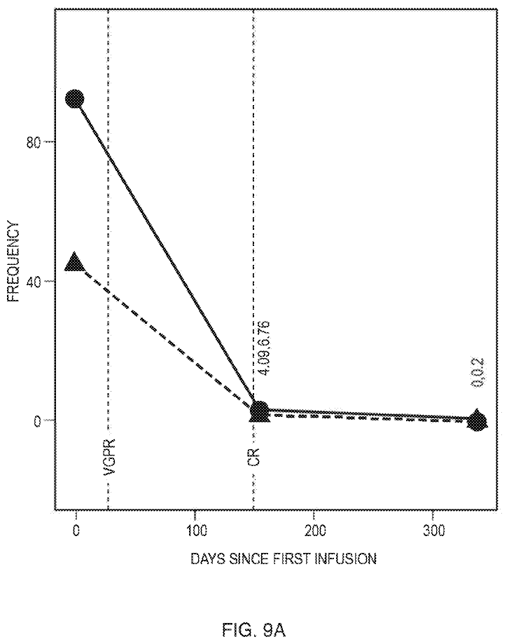

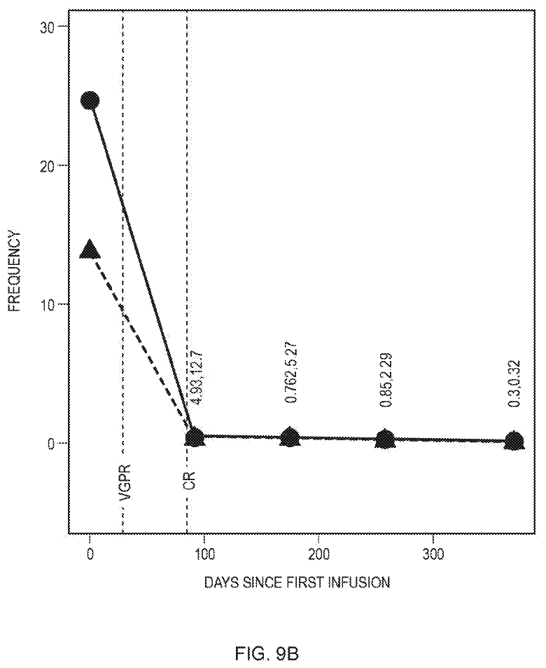

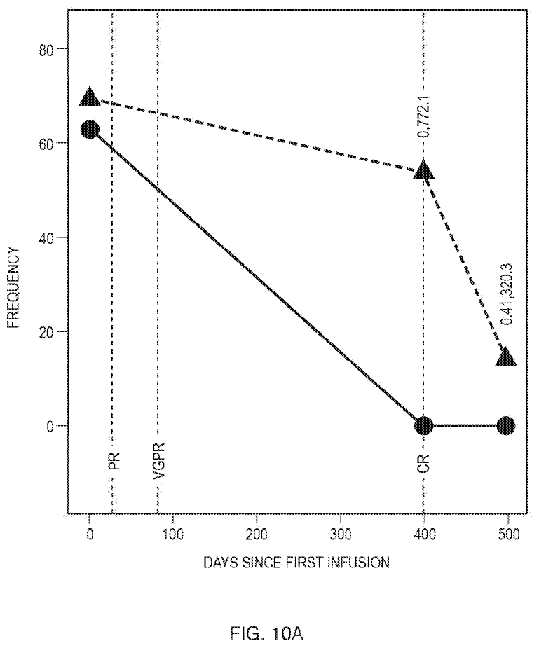

Some subjects showed rapid clinical response as well as a strong reduction in the clone frequency, but did not reach MRD negativity at first evaluation at the time of suspected CR. These subjects required more time to reach MRD negativity, but did eventually become MRD negative at later time points. FIG. 9A and FIG. 9B show the MRD profiles of two such subjects.

### Slow Responders

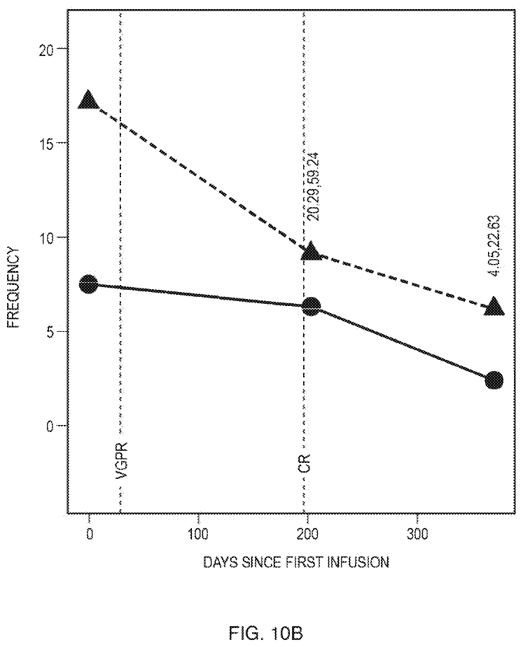

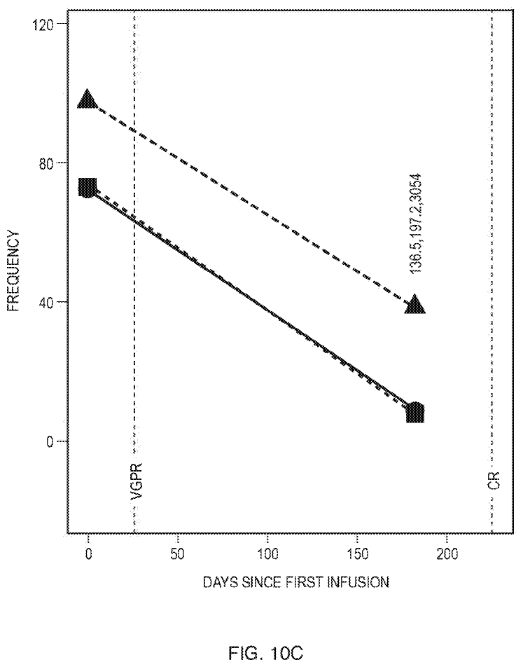

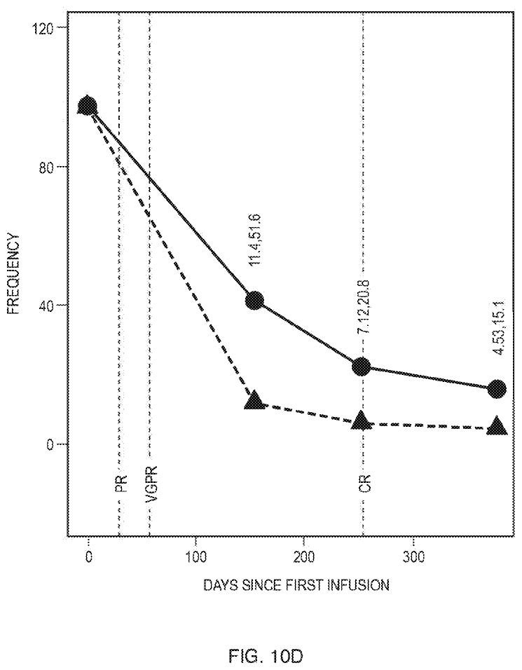

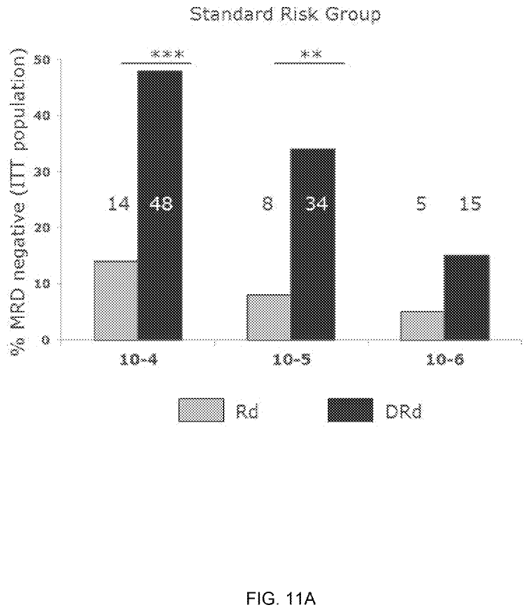

A number of subjects were identified who reached clinical response status of CR or sCR in which the MRD individual clone receptors clearly decreased, interestingly some at different rates, but who remained MRD positive. FIG. 10A, FIG. 10B, FIG. 10C and FIG. 10D show the MRD profiles of four such subjects.

### MRD Negativity in High-Risk and Standard Risk Subjects

The analysis sets included subgroup analyses of subjects from the POLLUX (Example 2) and CASTOR (Example 1) trials who were classified as high-risk or standard risk subjects based on cytogenetics status as described herein. MRD negativity was assessed separately at thresholds 10⁻⁴, 10⁻⁵ and 10⁻⁶.

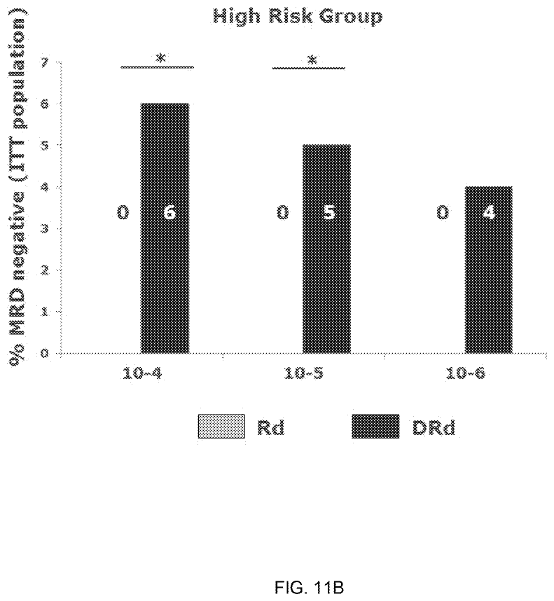

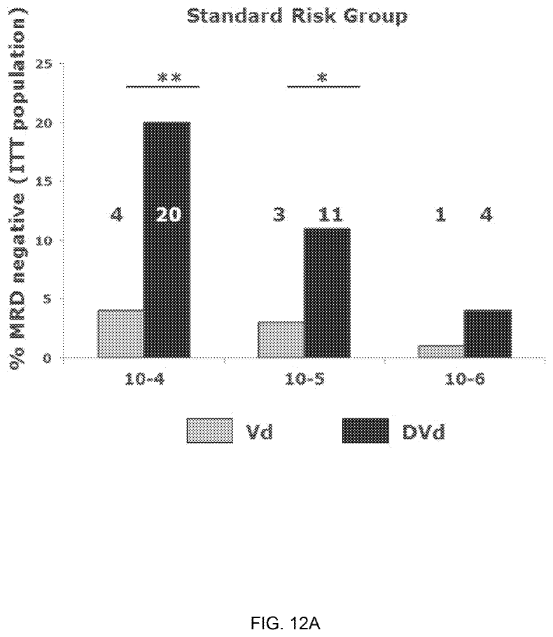

In POLLUX, significantly higher percentage of standard risk subjects achieved MRD negativity at 10⁻⁴ (p<0.0001; 48 pts vs 14 pts DRd vs Rd) and at 10⁻⁵ (p<0.005; 34 pts vs 8 pts DRd vs Rd) MRD threshold (FIG. 11A). No statistical significance was reached at 10⁻⁶ MRD threshold in standard risk subjects (15 pts vs 5 pts DRd vs Rd). Significantly higher percentage of high risk subjects achieved MRD negativity at 10⁻⁴ (p<0.05; 6 pts vs 0 pts DRd vs Rd) and at 10⁻⁵ (p<0.05; 5 pts vs 0 pts DRd vs Rd) MRD threshold (FIG. 11B). No statistical significance was reached at 10⁻⁶ MRD threshold in standard risk subjects (4 pts vs 0 pts DRd vs Rd).

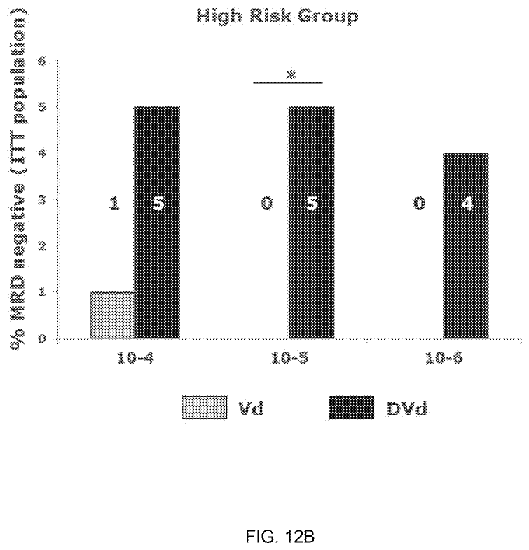

In CASTOR, significantly higher percentage of standard risk subjects achieved MRD negativity at 10⁻⁴ (p<0.005; 20 pts vs 4 pts DVd vs Vd) and at 10⁻⁵ (p<0.05; 11 pts vs 3 pts DVd vs Vd) MRD thresholds (FIG. 12A). No statistical significance was reached at 10⁻⁶ MRD threshold in standard risk subjects (4 pts vs 1 pts DVd vs Vd). Significantly higher percentage of high risk subjects achieved MRD negativity at 10⁻⁵ (p<0.05; 5 pts vs 0 pts DVd vs Vd) MRD threshold (FIG. 12B). No statistical significance was reached at 10⁻⁴ (5 pts vs 1 pts DVd vs Vd) or at 10⁻⁶ (4 pts vs 0 pts DVd vs Vd) MRD thresholds in high risk subjects (4 pts vs 0 pts DRd vs Rd).

### MRD Case Type: Conclusions

These plots highlight the potential utility of longitudinal MRD analysis to further categorize response and escape phenotypes. In addition, patients may be identified as to who will progress on therapy and may allow for earlier treatment interventions of next lines of therapy.

### CONCLUSION

Minimal residual disease negativity rate was significantly higher in the daratumumab containing regimens compared to the control groups (DRd: 29% vs Rd: 7.8% in Study POLLUX and DVd: 14% vs Vd: 3.0% in Study CASTOR) at the predefined hierarchical threshold of 10⁻⁴ and at least a 3-fold greater MRD negativity rate regardless of background therapies or threshold. Additional evaluation at the more stringent threshold of 10⁻⁵ demonstrated that daratumumab containing regimens also reached significantly higher MRD negativity rates as compared to the control groups. Further, these MRD data highlight the ability of daratumumab containing regimens in driving deep responses in this challenging patient population. Also at 10⁻⁶, DVd induced increased MRD negative rates compared to Vd, although statistical significance was not reached.

In summary, these two studies represent the first randomized, controlled, prospective evaluation of MRD in the relapsed or refractory MM Phase 3 clinical study setting and demonstrated that daratumumab-containing therapies are able to remarkably induce deep levels of clinical response in MM subjects. Regardless of the background therapies, daratumumab-containing regimens consistently showed 3-fold or greater increases in MRD negativity rate compared with the control groups at all evaluated thresholds. Importantly, since subjects who achieved MRD negative status demonstrated low PFS event rates, the deep clinical responses induced by addition of daratumumab may lead to improved long-term outcomes.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

TABLE 9
Sequences

| SEQ ID NO: | Sequence |
|---|---|
| Human CD38 aa | MANCEFSPVSGDKPCCRLSRRAQLCLGVSILVLILVVVLAVVVP |
| SEQ ID NO: 1 | RWRQQWSGPGTTKRFPETVLARCVKYTEIHPEMRHVDCQSVWD |
| | AFKGAFISKHPCNITEEDYQPLMKLGTQTVPCNKILLWSRIKD |
| | LAHQFTQVQRDMFTLEDTLLGYLADDLTWCGEFNTSKINYQSC |
| | PDWRKDCSNNPVSVFWKTVSRRFAEAACDVVHVMLNGSRSKIF |
| | DKNSTFGSVEVHNLQPEKVQTLEAWVIHGGREDSRDLCQDPTI |
| | KELESIISKRNIQFSCKNIYRPDKFLQCVKNPEDSSCTSEI |
| Human CD38 region | SKRNIQFSCKNIYR |
| 1 aa | |
| SEQ ID NO: 2 | |
| Human CD38 region | EKVQTLEAWVIHGG |
| 2 aa | |
| SEQ ID NO: 3 | |
| Daratumumab VH | EVQLLESGGGLVQPGGSLRLSCAVSGFTFNSFAMSWVRQAPGK |
| SEQ ID NO: 4 | GLEWVSAISGSGGGTYYADSVKGRFTISRDNSKNTLYLQMNSL |
| | RAEDTAVYFCAKDKILWFGEPVFDYWGQGTLVTVSS |
| Daratumumab VL | EIVLTQSPATLSLSPGERATLSCRASQSVSSYLAWYQQKPGQAP |
| SEQ ID NO: 5 | RLLIYDASNRATGIPARFSGSGSGTDFTLTISSLEPEDFAVYYC |
| | QQRSNWPPTFGQGTKVEIK |
| Daratumumab | SFAMS |
| HCDR1 | |
| SEQ ID NO: 6 | |
| Daratumumab | AISGSGGGTYYADSVKG |
| HCDR2 | |
| SEQ ID NO: 7 | |
| Daratumumab | DKILWFGEPVFDY |
| HCDR3 | |
| SEQ ID NO: 8 | |
| Daratumumab | RASQSVSSYLA |
| LCDR1 | |
| SEQ ID NO: 9 | |
| Daratumumab | DASNRAT |
| LCDR2 | |
| SEQ ID NO: 10 | |
| Daratumumab | QQRSNWPPTF |
| LCDR3 | |
| SEQ ID NO: 11 | |
| Daratumumab HC | EVQLLESGGGLVQPGGSLRLSCAVSGFTFNSFAMSWVRQAPGK |
| SEQ ID NO: 12 | GLEWVSAISGSGGGTYYADSVKGRFTISRDNSKNTLYLQMNSL |
| | RAEDTAVYFCAKDKILWFGEPVFDYWGQGTLVTVSSASTKGPS |
| | VFPLAPSSKSTSGGTAALGCLVKDYFPEPVTVSWNSGALTSGVH |
| | TFPAVLQSSGLYSLSSVVTVPSSSLGTQTYICNVNHKPSNTKVD |
| | KRVEPKSCDKTHTCPPCPAPELLGGPSVFLFPPKPKDTLMISRT |
| | PEVTCVVVDVSHEDPEVKFNWYVDGVEVHNAKTKPREEQYNST |
| | YRVVSVLTVLHQDWLNGKEYKCKVSNKALPAPIEKTISKAKGQ |
| | PREPQVYTLPPSREEMTKNQVSLTCLVKGFYPSDIAVEWESNG |
| | QPENNYKTTPPVLDSDGSFFLYSKLTVDKSRWQQGNVFSCSVM |
| | HEALHNHYTQKSLSLSPGK |
| Daratumumab LC | EIVLTQSPATLSLSPGERATLSCRASQSVSSYLAWYQQKPGQAP |
| SEQ ID NO: 13 | RLLIYDASNRATGIPARFSGSGSGTDFTLTISSLEPEDFAVYYC |
| | QQRSNWPPTFGQGTKVEIKRTVAAPSVFIFPPSDEQLKSGTASV |
| | VCLLNNFYPREAKVQWKVDNALQSGNSQESVTEQDSKDSTYSLS |
| | STLTLSKADYEKHKVYACEVTHQGLSSPVTKSFNRGEC |
| mAb003 VH | QVQLVQSGAEVKKPGSSVKVSCKASGGTFSSYAFSWVRQAPGQ |
| SEQ ID NO: 14 | GLEWMGRVIPFLGIANSAQKFQGRVTITADKSTSTAYMDLSSL |
| | RSEDTAVYYCARDDIAALGPFDYWGQGTLVTVSSAS |
| mAb003 VL | DIQMTQSPSSLSASVGDRVTITCRASQGISSWLAWYQQKPEKAP |
| SEQ ID NO: 15 | KSLIYAASSLQSGVPSRFSGSGSGTDFTLTISSLQPEDFATYYC |
| | QQYNSYPRTFGQGTKVEIK |
| mAb024 VH | EVQLVQSGAEVKKPGESLKISCKGSGYSFSNYWIGWVRQMPGK |
| SEQ ID NO: 16 | GLEWMGIIYPHDSDARYSPSFQGQVTFSADKSISTAYLQWSSL |
| | KASDTAMYYCARHVGWGSRYWYFDLWGRGTLVTVSS |
| mAb024 VL | EIVLTQSPATLSLSPGERATLSCRASQSVSSYLAWYQQKPGQAP |
| SEQ ID NO: 17 | GLLIYDASNRASGIPARFSGSGSGTDFTLTISSLEPEDFAVYYC |
| | QQRSNWPLTFGGGTKVEIK |
| MOR-202 VH | QVQLVESGGGLVQPGGSLRLSCAASGFTFSSYYMNWVRQAPG |
| SEQ ID NO: 18 | KGLEWVSGISGDPSNTYYADSVKGRFTISRDNSKNTLYLQMNS |
| | LRAEDTAVYYCARDLPLVYTGFAYWGQGTLVTVSS |
| MOR-202 VL | DIELTQPPSVSVAPGQTARISCSGDNLRHYYVYWYQQKPGQAP |
| SEQ ID NO: 19 | VLVIYGDSKRPSGIPERFSGSNSGNTATLTISGTQAEDEADYY |
| | CQTYTGGASLVFGGGTKLTVLGQ |
| Isatuximab VH | QVQLVQSGAEVAKPGTSVKLSCKASGYTFTDYWMQWVKQRPG |
| SEQ ID NO: 20 | QGLEWIGTIYPGDGDTGYAQKFQGKATLTADKSSKTVYMHLS |
| | SLASEDSAVYYCARGDYYGSNSLDYWGQGTSVTVSS |
| Isatuximab VL | DIVMTQSHLSMSTSLGDPVSITCKASQDVSTVVAWYQQKPGQS |
| SEQ ID NO: 21 | PRRLIYSASYRYIGVPDRFTGSGAGTDFTFTISSVQAEDLAVY |
| | YCQQHYSPPYTFGGGTKLEIK |

What is claimed is:

1. A method of treating multiple myeloma in a subject that had received two prior therapies, comprising administering to the subject a combination regimen comprising a therapeutically effective amount of daratumumab, bortezomib, and dexamethasone,
wherein the subject achieves a complete response (CR);
wherein the subject is minimal residual disease (MRD) negative after at least 6 months of achieving the CR;
wherein the subject exhibits progression-free survival (PFS) of at least 16 months following initiation of the combination regimen; and
wherein the method increases PFS compared to administering dexamethasone and bortezomib without the daratumumab.

2. The method of claim 1, wherein the method increases overall response rate (ORR) compared to administering dexamethasone and bortezomib without the daratumumab.

3. The method of claim 2, wherein the method increases rate of very good partial response (VGPR) or better compared to administering dexamethasone and bortezomib without the daratumumab.

4. The method of claim 1, wherein the MRD is determined at a sensitivity threshold of 0.0001%.

5. The method of claim 1, wherein the subject has had autologous stem cell transplantation (ASCT).

6. The method of claim 1, wherein the subject achieves a stringent complete response.

7. The method of claim 1, wherein the subject had received a proteasome inhibitor as one of the two prior therapies.

8. The method of claim 7, wherein the proteasome inhibitor is bortezomib.

9. The method of claim 1, wherein the subject had received an immunomodulatory agent as one of the two prior therapies.

10. The method of claim 9, wherein the immunomodulatory agent is lenalidomide.

11. The method of claim 1, wherein the subject has relapsed or refractory multiple myeloma.

12. The method of claim 11, wherein the subject is refractory to lenalidomide.

13. The method of claim 11, wherein the subject is refractory to bortezomib.

14. A method of treating relapsed or refractory multiple myeloma in a subject that had received two prior therapies, comprising administering to the subject a combination regimen comprising a therapeutically effective amount of daratumumab, lenalidomide, and dexamethasone,
wherein the subject achieves a CR;
wherein the subject is MRD negative after at least 6 months of achieving the CR;
wherein the subject exhibits PFS of at least 21 months following initiation of the combination regimen;
and
wherein the method increases PFS compared to administering dexamethasone and lenalidomide without the daratumumab.

15. The method of claim 14, wherein the method increases ORR compared to administering dexamethasone and lenalidomide without the daratumumab.

16. The method of claim 15, wherein the method increases rate of VGPR or better compared to administering dexamethasone and lenalidomide without the daratumumab.

17. The method of claim 15, wherein the method increases rate of CR or better compared to administering dexamethasone and lenalidomide without the daratumumab.

18. The method of claim 14, wherein the MRD is determined at a sensitivity threshold of 0.0001%.

19. The method of claim 14, wherein the subject has had ASCT.

20. The method of claim 14, wherein the subject had received a proteasome inhibitor as one of the two prior therapies.

21. The method of claim 20, wherein the proteasome inhibitor is bortezomib.

22. The method of claim 14, wherein the subject had received an immunomodulatory agent as one of the two prior therapies.

23. The method of claim 22, wherein the immunomodulatory agent is lenalidomide.

24. The method of claim 14, wherein the subject is refractory to lenalidomide.

25. The method of claim 14, wherein the subject is refractory to bortezomib.


SEQUENCE LISTING (21 sequences)

SEQ ID NO: 1 - protein, 300 aa, Homo sapiens
  REGION 1..300: note = Human CD38 aa
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 14 aa, Homo sapiens
  REGION 1..14: note = Human CD38 region 1 aa
SKRNIQFSCKNIYR

SEQ ID NO: 3 - protein, 14 aa, Homo sapiens
  REGION 1..14: note = Human CD38 region 2 aa
EKVQTLEAWVIHGG

SEQ ID NO: 4 - protein, 122 aa, synthetic construct [sequence omitted]

SEQ ID NO: 5 - protein, 107 aa, synthetic construct [sequence omitted]

SEQ ID NO: 6 - protein, 5 aa, synthetic construct
  REGION 1..5: note = Daratumumab HCDR1
SFAMS

SEQ ID NO: 7 - protein, 17 aa, synthetic construct
  REGION 1..17: note = Daratumumab HCDR2
AISGSGGGTYYADSVKG

SEQ ID NO: 8 - protein, 13 aa, synthetic construct
  REGION 1..13: note = Daratumumab HCDR3
DKILWFGEPVFDY

SEQ ID NO: 9 - protein, 11 aa, synthetic construct
  REGION 1..11: note = Daratumumab LCDR1
RASQSVSSYLA

SEQ ID NO: 10 - protein, 7 aa, synthetic construct
  REGION 1..7: note = Daratumumab LCDR2
DASNRAT

SEQ ID NO: 11 - protein, 10 aa, synthetic construct
  REGION 1..10: note = Daratumumab LCDR3
QQRSNWPPTF

SEQ ID NO: 12 - protein, 452 aa, synthetic construct
  REGION 1..452: note = Daratumumab HC
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
  REGION 1..214: note = Daratumumab LC
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 122 aa, synthetic construct
  REGION 1..122: note = mAb003 VH
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSAS

SEQ ID NO: 15 - protein, 107 aa, synthetic construct
  REGION 1..107: note = mAb003 VL
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 16 - protein, 122 aa, synthetic construct
  REGION 1..122: note = mAb024 VH
EVQLVQSGAEVKKPGESLKISCKGSGYSFSNYWIGWVRQMPGKGLEWMGIIYPHDSDARYSPSFQGQVTFSADKSISTAYLQWSSLKASDTAMYYCARHVGWGSRYWYFDLWGRGTLVTVSS

SEQ ID NO: 17 - protein, 107 aa, synthetic construct
  REGION 1..107: note = mAb024 VL
EIVLTQSPATLSLSPGERATLSCRASQSVSSYLAWYQQKPGQAPGLLIYDASNRASGIPARFSGSGSGTDFTLTISSLEPEDFAVYYCQQRSNWPLTFGGGTKVEIK

SEQ ID NO: 18 - protein, 120 aa, synthetic construct
  REGION 1..120: note = MOR-202 VH
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 19 - protein, 109 aa, synthetic construct
  REGION 1..109: note = MOR-202 VL
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 20 - protein, 120 aa, synthetic construct [sequence omitted]

SEQ ID NO: 21 - protein, 107 aa, synthetic construct [sequence omitted]